(19) United States
(12) Patent Application Publication
(10) Pub. No.: US 2026/0055210 A1
(43) Pub. Date: Feb. 26, 2026

(54) FLT3 LIGAND BI-FUNCTIONAL MOLECULES FOR THROMBOPENIA AND ACUTE RADIATION SYNDROME

(71) Applicants: Montefiore Medical Center, Bronx, NY (US); Albert Einstein College of Medicine, Bronx, NY (US)

(72) Inventors: Chandan GUHA, Scarsdale, NY (US); Steven ALMO, Pelham, NY (US); Phaneendra DUDDEMPUDI, Hillsdale, NJ (US)

(21) Appl. No.: 19/105,590

(22) PCT Filed: Aug. 23, 2023
(86) PCT No.: PCT/US2023/072760

§ 371 Date: Feb. 21, 2025

Related U.S. Application Data
(60) Provisional application No. 63/373,436, filed on Aug. 24, 2022.

(51) Int. Cl. C07K 16/40; A61K 38/00; A61P 35/00; C07K 14/475; C07K 14/52; C12N 15/62; C12N 15/85

(52) U.S. Cl. CPC C07K 16/40; A61K 38/00; A61P 35/00; C07K 14/475; C07K 14/524; C12N 15/62; C12N 15/85; C07K 2317/32; C07K 2317/34; C07K 2317/52; C07K 2317/77; C07K 2319/50

(57) ABSTRACT

Provided herein are polypeptides, compositions, and methods for treating a cancer in an individual using a polypeptide comprising a thrombopoietin domain and a Flt3 ligand domain. Also provided herein are nucleic acids encoding such polypeptides, expression vectors and cells comprising such nucleic acids, and methods of producing the polypeptides comprising a thrombopoietin domain and a Flt3 ligand domain. The administration of a fusion polypeptide comprising a thrombopoietin domain and a Flt3 ligand domain to a subject may treat and reduce the symptoms of hematopoietic failure, including thrombopenia, and/or acute radiation syndrome.

### CROSS-REFERENCE

This application claims the benefit of U.S. Provisional Application No. 63/373,436, filed on Aug. 24, 2022, which is incorporated herein by reference in its entirety.

### BACKGROUND

Among the deleterious effects of exposure to high doses of radiation are hematopoietic failure, thrombocytopenia, and acute radiation syndrome. In some cases, the toxicity of radiation treatment may limit the radiation doses for cancer patients. In some cases, a subject may have a condition resulting in hematopoietic failure. Fms-like tyrosine kinase 3 (Flt3) ligand (FL) and thrombopoietin have been envisioned as possible treatments to counteract the deleterious effects of radiation exposure and/or to increase hematopoietic activity. However, the effectiveness of FL and thrombopoietin may be limited by short half-life in circulation.

### SUMMARY

Provided herein are polypeptides, compositions, and methods for supporting cancer treatment in an individual using a polypeptide comprising a thrombopoietin domain and a Flt3 ligand domain. Also described herein are nucleic acids encoding such polypeptides, expression vectors and cells comprising such nucleic acids, and methods of producing the polypeptides comprising a thrombopoietin domain and a Flt3 ligand domain.

Described herein are polypeptides comprising a thrombopoietin domain and a Flt3 ligand domain. In some embodiments, the thrombopoietin domain comprises an amino acid sequence at least 80%, 85%, 90%, 95%, or 100% identical to SEQ ID NO: 9. In some embodiments, the Flt3 ligand domain comprises an amino acid sequence at least 80%, 85%, 90%, 95%, or 100% identical to SEQ ID NO: 3 or 5. Described herein are polypeptides comprising an amino acid sequence at least 80%, 85%, 90%, 95%, or 100% identical to SEQ ID NO: 1. In some embodiments, the polypeptide further comprises an immunoglobulin Fc polypeptide or a fragment thereof. In some embodiments, the immunoglobulin is immunoglobulin G1 (IgG1). In some embodiments, the polypeptide further comprises an EPO leader sequence and/or a TEV cleavage domain. In some embodiments, the polypeptide further comprises a linker. In some embodiments, the linker couples the amino acid at least 80% identical to SEQ ID NO: 9 to the immunoglobulin Fc polypeptide or a fragment thereof. In some embodiments, the linker couples the thrombopoietin domain to the immunoglobulin Fc polypeptide or a fragment thereof. In some embodiments, the linker couples the thrombopoietin domain to a second thrombopoietin domain. In some embodiments, the linker couples the TEV domain to the thrombopoietin domain or the second thrombopoietin domain. In some embodiments, the linker couples the amino acid at least 80% identical to SEQ ID NO: 3 or 5 to the immunoglobulin Fc polypeptide or a fragment thereof. In some embodiments, the linker couples the Flt3 ligand domain to the immunoglobulin Fc polypeptide or a fragment thereof. In some embodiments, the Flt3 ligand domain is a Flt3 ligand isoform 1. In some embodiments, the Flt3 ligand domain is a human Flt3 ligand isoform 1. In some embodiments, the amino acid sequence is identical to SEQ ID NO: 1. In some embodiments, the polypeptide is an immunoglobulin Fc polypeptide or fragment thereof and comprises one or more alternations compared to a wild type IgG Gc region as specific in SEQ ID NO:7. In some embodiments the one or more alterations affects an immunological property of the immunoglobulin Fc polypeptide or a fragment thereof. In some embodiments, the one or more alterations comprises L234A, L235A, N297A, N297Q, P329Q, or a combination thereof according to EU numbering. In some embodiments, the one or more alterations comprises L234A and L235A according to EU numbering. In some embodiments, the one or more alterations comprises N297A according to EU numbering. In some embodiments, the one or more alterations comprises N297Q according to EU numbering. In some embodiments, the one or more alterations comprises P329Q according to EU numbering. In some embodiments, the immunological property comprises antigen-dependent cellular cytotoxicity (ADCC), complement dependent cytotoxicity (CDC), antibody-dependent cell-mediated phagocytosis (ADCP), or a combination thereof. In some embodiments, an administration of the polypeptide to a subject increases a number of platelets in the subject. In some embodiments, an administration of the polypeptide to a subject increases a number of dendritic cells in blood of the subject. In some embodiments, the polypeptide improves survival of cells associated with hematopoiesis in treated cells as compared to untreated cells. In some embodiments, the polypeptide stimulates proliferation of cells associated with hematopoiesis in treated cells as compared to untreated cells. In some embodiments, the polypeptide activates MAPK pathway. In some embodiments, the polypeptide increases ERK phosphorylation in treated cells as compared to untreated cells. In some embodiments, the polypeptide increases Erk1/2 phosphorylation in treated cells as compared to untreated cells. In some embodiments, the polypeptide activates PI3K/Akt pathway. In some embodiments, the polypeptide increases Akt phosphorylation in treated cells as compared to untreated cells. In some embodiments, the polypeptide increases ERK/Akt phosphorylation in treated cells as compared to untreated cells. In some embodiments, the polypeptide is configured to bind to a fms like tyrosine kinase 3 (FLT3). Provided herein are compositions comprising the polypeptide provided herein and a pharmaceutically acceptable excipient. In some embodiments, the composition is formulated to be administered either intravenously or subcutaneously or intra-tumorally.

Provided herein are methods of supporting cancer treatment in an individual, the method comprising administering any one of the polypeptides provided herein to the individual. Provided herein are methods of supporting cancer treatment in an individual, the method comprising administering any one of the compositions provided herein to the individual. Provided herein are methods of modulating an immune response in an individual, the method comprising administering any one of the polypeptides provided herein to the individual. Provided herein are methods of stimulating the proliferation and activation of dendritic cells, the method comprising administering any one of the polypeptides provided herein to an individual. In some embodiments, the method improves survival of cells associated with hematopoiesis in treated cells as compared to untreated cells. In some embodiments, the method stimulates proliferation of cells associated with hematopoiesis in treated cells as compared to untreated cells. In some embodiments, the method activates MAPK pathway. In some embodiments, the method increases ERK phosphorylation in treated cells as compared to untreated cells. In some embodiments, the method increases Erk1/2 phosphorylation in treated cells as compared to untreated cells. In some embodiments, the method activates PI3K/Akt pathway. In some embodiments, the method increases Akt phosphorylation in treated cells as compared to untreated cells. In some embodiments, the method increases ERK/Akt phosphorylation in treated cells as compared to untreated cells.

Provided herein are nucleic acids encoding any one of the polypeptides provided herein. Described herein are expression vectors comprising any one of the nucleic acids provided herein. Described herein are cells comprising the nucleic acid encoding any one of the polypeptides provided herein. Described herein are methods of producing any one of polypeptides provided herein comprising culturing cells under conditions sufficient to express the polypeptide. Described herein are any one of the polypeptides provided herein for use in a method of supporting cancer treatment in an individual.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows a schematic of an exemplary fusion polypeptide comprising a FL domain, a human IgG1 domain, and a thrombopoietin domain.

FIG. 2 illustrates 4-12% Bis-Tris gel showing the expression of fusion polypeptides comprising a FL domain and a thrombopoietin domain, indicated with an arrow.

FIG. 3 illustrates the non-reduced and reduced purified FLT3L-1-Romiplostim protein on 4-12% Bis-Tris precast SDS-PAGE gel. SDS-PAGE gel that shows a protein band at approximately 100 kDa under non-reduced condition and approximately 70 kDa under reduced condition (indicated with an arrow), which corresponds to the predicted molecular weight of the FL-Romiplostim fusion polypeptide under each condition. The first lane shows the unstained protein ladder.

FIG. 4 shows the flow cytometry results showing FL-romiplostim fusion polypeptide rescue from apoptosis in OCI-AML5 cells starved of FBS and growth factors.

FIG. 5 shows the percentage of apoptotic cells from the flow cytometry results.

FIGS. 6A and 6B show the percentage of cells positive and mean fluorescence intensity for phosphorylated Erk1/2 proteins from the flow cytometry results.

FIGS. 7A and 7B show the percentage of cells positive for total Erk1/2 expression and mean fluorescence intensity using p44/42 MAPK (Erk1/2) antibody from the flow cytometry results, providing a baseline for Erk1/2 expression of about 70-75% percent positive cells.

FIGS. 8A and 8B show the percentage of cells positive and mean fluorescence intensity for PI3K-Akt phosphorylation from the flow cytometry results.

FIGS. 9A and 9B show the percentage of cells positive and mean fluorescence intensity for PI3K-Akt expression using Akt (pan) (C67E7) antibody from the flow cytometry results, providing a baseline for Akt expression of about 80-90% percent positive cells.

FIGS. 10A and 10B show the percentage of cells positive and mean fluorescence intensity for Erk1/2 phosphorylation after treatment with thrombopoietin mimetic (TPOm) peptide.

FIGS. 11A and 11B show the percentage of cells positive and mean fluorescence intensity for Erk1/2 phosphorylation after treatment with romiplostim.

FIGS. 12A and 12B show the percentage of cells positive and mean fluorescence intensity for Erk1/2 phosphorylation from the flow cytometry results.

FIG. 13 shows the results of the XTT assay of the M-07e cell line cultured with the FL-romiplostim fusion polypeptide and assessed for its effect on cell proliferation.

FIG. 14 shows an example of the results of the binding of the FLT3L-Romiplostim fusion polypeptide to human cMLP or mouse cMLP expressing cells.

FIG. 15 illustrates an example of a 4-12% Bis-Tris gel showing the expression of FLT3L-Romiplostim fusion polypeptides Fc mutants.

FIG. 16 shows an example of the increase in plasma concentration via subcutaneous injection of FLT3L-Romiplostim Fc mutants compared to wild-type Fc or controls.

FIG. 17 shows an example of the increase in plasma concentration via intravenous injection of FLT3L-Romiplostim Fc mutants compared to controls.

FIG. 18 shows an example of the increase in platelet counts from injection with FLT3L-Romiplostim over controls.

FIGS. 19A and 19B show an example of the increase in platelet spleen and blood DCs, respectively, from injection with FLT3L-Romiplostim over controls.

### DETAILED DESCRIPTION

An individual having a hematopoietic failure, also referred to as bone marrow failure (BMF), may have a decreased production of one or more cell types in the hematopoietic lineages. Usually, hematopoietic failure may result in a reduced number of hematopoietic precursors in the bone marrow of the individual and cytopenia. In some cases, the hematopoietic failure may be inherited or acquired. In some cases, exposure to high doses of radiation may result in an acquired hematopoietic failure, including but not limited to thrombocytopenia, and acute radiation syndrome. In some cases, cancer patients undergoing radiation treatment may experience severe side effects, including radiation syndrome, thrombocytopenia, and hematopoietic failures. In some cases, the toxicity of radiation treatment may limit the amount of radiation treatment the patient may be able to receive and may make completion of a treatment regimen challenging and reduction of the tumor difficult. Reducing the effects of radiation syndrome, thrombocytopenia, and hematopoietic failures may allow cancer patients to receive more radiation treatment. In some cases, an individual may have an acute, high-dose radiation exposure that may result in adverse health outcomes, such as radiation toxicity and acute radiation syndrome.

Thrombocytopenia is a condition that occurs when the platelet count is too low. As platelets are plays a critical role in helping blood to clot, thrombocytopenia is sometimes associated with abnormal bleeding. In some cases, thrombocytopenia may result from decreased production of platelets in the bone marrow, increased breakdown of platelets in the bloodstream, and/or increased breakdown of platelets in the spleen or liver.

Fms-like tyrosine kinase 3 (Flt3) ligand (FL) and thrombopoietin have been envisioned as possible treatments to counteract the deleterious effects of radiation exposure and/or increase hematopoietic activity and/or recovery. In some cases, injections of FL have shown protective effect from high doses of radiation, including but not limited to increased myelopoietic activity and hematopoietic recovery. Often, thrombopoietin may be used to increase the number of platelets in order to decrease the risk of bleeding in individual who have thrombocytopenia. However, the effectiveness of FL and thrombopoietin may be limited by short half-life in circulation. Usually, FL has a half-life of less than five hours after an intraperitoneal injection in a mouse model.

Often, the half-life may be extended when FL is presented as a fusion polypeptide, also referred herein as a fusion protein. In some cases, a human FL-fragment crystallizable (Fc) fusion polypeptide has a half-life of about 24 hours in a mouse model. Usually, romiplostim, a Fc-peptide fusion protein (peptibody) that an analog of thrombopoietin (TPO), has a half-life ranging from 1 to 34 days with a median of about 3.5 days. Sometimes, the presence of a Fc domain increases the half-life of a polypeptide. In some cases, the increased half-life may be due to the interaction of the Fc domain with a neonatal Fc-receptor, which aid in the recycling of endocytosed Fc fusion polypeptide. In some cases, the presence of a Fc domain allows for a cost-effective, single-step purification of fusion polypeptides. In some cases, the presence of a Fc domain improves the solubility and stability of the partnered domain(s) in the fusion polypeptides. In some cases, the Fc domain comprises one or more alterations compared to a wild type IgG Fc region. In some cases, the one or more alterations affects an immunological property of the Fc domain, including but not limited to antigen-dependent cellular cytotoxicity (ADCC), complement dependent cytotoxicity (CDC), and antibody-dependent cell-mediated phagocytosis (ADCP).

Provided herein are polypeptides, compositions, and methods for treating a cancer in an individual using a polypeptide comprising a thrombopoietin domain and a FL domain. Also provided herein are nucleic acids encoding such polypeptides, expression vectors and cells comprising such nucleic acids, and methods of producing the polypeptides comprising a thrombopoietin domain and a FL domain. The administration of a fusion polypeptide comprising a thrombopoietin domain and a FL domain to a subject may treat and reduce the symptoms of hematopoietic failure, including thrombocytopenia, and/or acute radiation syndrome.

### Polypeptides

Provided herein are polypeptides comprising a thrombopoietin domain and a Flt3 ligand domain.

Fms-related tyrosine kinase 3 (Flt3) ligand (FL) is a hematopoietic cytokine that is encoded by the FLT3LG gene in humans and regulates proliferation of hematopoietic progenitor cells. Flt3 ligand (FL) binds to fms-like tyrosine kinase receptor Flt3/Flk2. FL is a homodimer of protomers composed of a four helix bundle and is structurally homologous to stem cell factor (SCF) and colony stimulating factor 1 (CSF-1). While FL does not stimulate proliferation of early hematopoietic cells by itself, FL synergizes with other CSFs and interleukins to induce growth and differentiation of various blood cell progenitors and may act a major growth factor stimulating the growth of dendritic cells. Multiple isoforms of FL have been identified, including a transmembrane isoform and a membrane-bound isoform. The predominant biologically active form (209 a.a.) is anchored to the cell surface with an extracellular domain of a transmembrane protein. The membrane-bound isoform can be proteolytically cleaved to generate a biologically active soluble isoform. In some cases, the active form of FL refers to the physiologically active form that is membrane anchored. In some cases, the active form of FL refers to the therapeutically active form that lacks the transmembrane segment.

Thrombopoietin (TPO), also known as megakaryocyte growth and development factor (MGDF), is a protein that in humans is encoded by the THPO gene. TPO is produced by the liver and kidney and regulates the production of platelets by stimulating the production and differentiation of megakaryocytes. TPO is a ligand for MLP/C_MPL, the product of myeloproliferative leukemia virus oncogene. In some cases, the plasma TPO level may be inversely correlated to the mass of megakaryocytes and platelets, which degrade the TPO following its binding to specific membrane receptors. In some cases, a function of a TPO or a TPO domain may be assessed by ELISA or a TPO performance assay, such as bead-based multiplex assays. In some embodiments, a thrombopoietin domain herein refers to thrombopoietin or a functional fragment thereof, or romiplostim or a functional fragment thereof. Romiplostim is a Fc-fusion protein functional analog of thrombopoietin that increases platelet production through activation of the thrombopoietin receptor. Romiplostim is a dimer Fc-peptide fusion protein (peptibody) that has two identical single-chain subunits, each one made up of 269 amino acid (a.a.) residues. Each subunit consists of a human IgG1 Fc carrier domain that is covalently attached to a polypeptide sequence that contains two binding domains to interact with thrombopoietin receptor c-Mpl. Each of the binding domains consists of 14 a.a. The amino acid sequence of romiplostim is not similar to that of endogenous thrombopoietin. When romiplostim binds to the TPO receptors, romiplostim may promote the growth of bone marrow megakaryocyte colony-forming cells, which leads to increased platelet production via JAK2 and STAT5 kinase pathways. Romiplostim may be used to treat low blood platelet counts (thrombocytopenia) and help prevent bleeding in patients with idiopathic thrombocytopenia (ITP). In some cases, romiplostim may be used to mitigate the effects of acute radiation syndrome (ARS). In some cases, romiplostim acts through similar pathways as thrombopoietin. In some embodiments, romiplostim comprises a thrombopoietin domain with to a Fc domain.

In some embodiments, the thrombopoietic domain comprises a functional analog or mimetic of thrombopoietin with a substantially same function thereof. In some embodiments, the thrombopoietic domain comprises a functional analog or mimetic of romiplostim with a substantially same function thereof. In some embodiments, when the thrombopoietin domain binds to a TPO receptor, the thrombopoietin domain may promote the growth of bone marrow megakaryocyte colony-forming cells. In some embodiments, when the thrombopoietin domain binds to a TPO receptor, the thrombopoietin domain may lead to increased platelet production. In some embodiments, the platelet production may increase via JAK2 and STAT5 kinase pathways. In some embodiments, administration of a composition comprising the thrombopoietin domain may treat thrombocytopenia and help prevent bleeding in patients with ITP. In some embodiments, administration of a composition the thrombopoietin domain. In some embodiments, a function of a thrombopoietin domain may be assessed by ELISA or a TPO performance assay, such as bead-based multiplex assays.

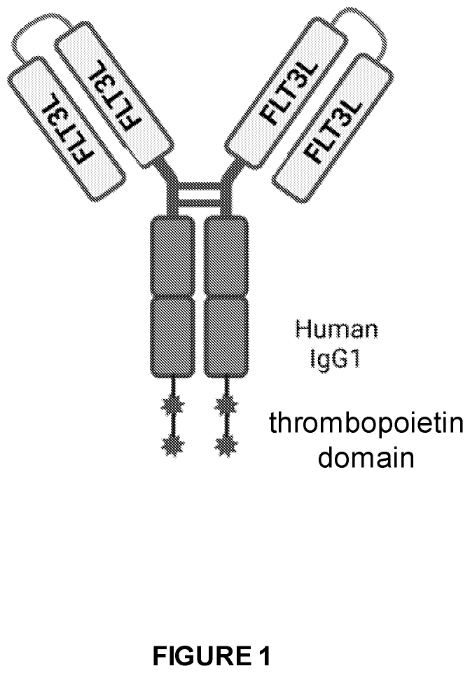

Provided herein are polypeptides, compositions, and methods for supporting cancer treatment in an individual using a polypeptide comprising a thrombopoietin domain and a Flt3 ligand domain. Also provided herein are nucleic acids encoding such polypeptides, expression vectors and cells comprising such nucleic acids, and methods of producing the polypeptides comprising a thrombopoietin domain and a Flt3 ligand domain. The administration of a fusion polypeptide comprising a thrombopoietin domain and a Flt3 ligand domain to a subject may treat and reduce the symptoms of hematopoietic failure, including thrombopenia, and/or acute radiation syndrome. FIG. 1 shows a schematic of an exemplary fusion polypeptide comprising a FL domain, a human IgG1 domain, and a thrombopoietin domain.

Provided herein are polypeptides comprising a human Flt3 ligand domain and a thrombopoietin domain. In some embodiments, the polypeptide is configured to bind to a fms like tyrosine kinase 3 (Flt3). In some embodiments, the polypeptide comprises a thrombopoietin domain and a Flt3 ligand domain. In some embodiments, the thrombopoietin domain comprises an amino acid sequence at least 30%, 35%, 40%, 45%, 50%, 55%, 60%, 65%, 70%, 75%, 80%, 85%, 90%, 95%, or 100% identical to SEQ ID NO: 9. In some embodiments, thrombopoietin domain comprises an amino acid sequence that is SEQ ID NO: 9. In some embodiments, the thrombopoietin domain comprises an amino acid sequence at least 30%, 35%, 40%, 45%, 50%, 55%, 60%, 65%, 70%, 75%, 80%, 85%, 90%, 95%, or 100% identical to romiplostim. In some embodiments, the Flt3 ligand domain comprises human Flt3 ligand isoform 1. In some embodiments, the Flt3 ligand isoform 1 is membrane bound. In some embodiments, the Flt3 ligand comprises a soluble Flt3 ligand. In some embodiments, the Flt3 ligand domain comprises an amino acid sequence at least 30%, 35%, 40%, 45%, 50%, 55%, 60%, 65%, 70%, 75%, 80%, 85%, 90%, 95%, or 100% identical to SEQ ID NO: 3. In some embodiments, the Flt3 ligand domain comprises an amino acid sequence at least 30%, 35%, 40%, 45%, 50%, 55%, 60%, 65%, 70%, 75%, 80%, 85%, 90%, 95%, or 100% identical to SEQ ID NO: 5. In some embodiments, the Flt3 ligand domain comprises an amino acid sequence that is SEQ ID NO: 3. In some embodiments, the Flt3 ligand domain comprises an amino acid sequence that is SEQ ID NO: 5. In some embodiments, the Flt3 ligand domain is a Flt3 ligand isoform 1. In some embodiments, the Flt3 ligand domain is a human Flt3 ligand isoform 1. In some embodiments, the amino acid sequence is identical to SEQ ID NO: 1.

In some embodiments, the polypeptide provided herein comprises an amino acid sequence at least 30%, 35%, 40%, 45%, 50%, 55%, 60%, 65%, 70%, 75%, 80%, 85%, 90%, 95%, or 100% identical to SEQ ID NO: 1. In some embodiments, the polypeptide comprises an amino acid sequence that is SEQ ID NO: 1.

In some embodiments, the polypeptide further comprises an immunoglobulin Fc polypeptide or a fragment thereof. In some embodiments, the immunoglobulin is immunoglobulin G (IgG). In some embodiments, the immunoglobulin is immunoglobulin G1 (IgG1). In some embodiments, the immunoglobulin comprises a human immunoglobulin isotype. In some embodiments, the immunoglobulin comprises one or more of IgG1, IgG2, IgG3, IgG4, IgA, IgE, or IgM. In some embodiments, the polypeptide comprises one or more alterations to the immunoglobulin Fc polypeptide or a fragment thereof compared to a wild type IgG Fc region as specified in SEQ ID NO: 7. In some embodiments, the one or more alterations comprises L234A, L235A, N297A, N297Q, P329Q, or a combination thereof according to EU numbering. In some embodiments, the one or more alterations comprises L234A and L235A (LALA) according to EU numbering. In some embodiments, the one or more alterations comprises N297A according to EU numbering. In some embodiments, the one or more alterations comprises N297Q according to EU numbering. In some embodiments, the one or more alterations comprises P329Q according to EU numbering. In some embodiments, the immunoglobulin comprises an effector function mutation. In some embodiments, the effector function mutation comprises L234A and L235A (LALA), N297A, N297Q, or P329Q or a combination thereof. In some embodiments, the immunoglobulin Fc polypeptide or a fragment thereof comprises an amino acid sequence at least 30%, 35%, 40%, 45%, 50%, 55%, 60%, 65%, 70%, 75%, 80%, 85%, 90%, 95%, or 100% identical to SEQ ID NO: 7.

In some embodiments, the polypeptide further comprises one or more signal sequences. In some embodiments, the polypeptide further comprises an EPO leader sequence and/or a tobacco etch virus (TEV) cleavage domain. In some embodiments, the signal sequence comprises an EPO leader sequence. In some embodiments, the signal sequence comprises an TEV cleavage domain. In some embodiments, the EPO leader sequence comprises an amino acid sequence at least 30%, 35%, 40%, 45%, 50%, 55%, 60%, 65%, 70%, 75%, 80%, 85%, 90%, 95%, or 100% identical to SEQ ID NO: 2. In some embodiments, the TEV cleavage domain comprises an amino acid sequence at least 30%, 35%, 40%, 45%, 50%, 55%, 60%, 65%, 70%, 75%, 80%, 85%, 90%, 95%, or 100% identical to SEQ ID NO: 10. In some embodiments, the signal sequence comprises one or more of human OSM, VSV-G, mouse Ig kappa, mouse Ig heavy chain, BM40, secrecon, human IgK VII, CD33, tPA, human chymotrypsinogen, human trypsinogen-2, Gaussia luc, albumin, influenza haemagglutinin, human insulin, or silkworm fibroin.

In some embodiments, the polypeptide further comprises a linker. In some embodiments, the linker couples the amino acid to the immunoglobulin Fc polypeptide or a fragment thereof. In some embodiments, the linker couples the amino acid at least 80% identical to SEQ ID NO: 3 or 5 to the immunoglobulin Fc polypeptide or a fragment thereof. In some embodiments, the linker couples the thrombopoietin domain to the immunoglobulin Fc polypeptide or a fragment thereof. In some embodiments, the link er couples the amino acid to the immunoglobulin Fc polypeptide or a fragment thereof is at least 80% identical to SEQ ID NO: 6. In some embodiments, the linker couples the Flt3 ligand domain to the immunoglobulin Fc polypeptide or a fragment thereof. In some embodiments, the linker coupling a Flt3 ligand domain to a second ligand domain is at least 80% identical to SEQ ID NO: 4. In some embodiments, the linker couples a Flt3 ligand domain to a second ligand domain. In some embodiments, the linker couples the thrombopoietin domain to a second thrombopoietin domain. In some embodiments, the linker couples the TEV domain to the thrombopoietin domain or the second thrombopoietin domain. In some embodiments, the linker couples the amino acid at least 80% identical to SEQ ID NO: 3 or 5 to the immunoglobulin Fc polypeptide or a fragment thereof. In some embodiments, the linker couples the Flt3 ligand domain to the immunoglobulin Fc polypeptide or a fragment thereof. In some embodiments, the linker comprises an amino acid sequence at least 30%, 35%, 40%, 45%, 50%, 55%, 60%, 65%, 70%, 75%, 80%, 85%, 90%, 95%, or 100% identical to SEQ ID NO: 4. In some embodiments, the linker comprises an amino acid sequence at least 30%, 35%, 40%, 45%, 50%, 55%, 60%, 65%, 70%, 75%, 80%, 85%, 90%, 95%, or 100% identical to SEQ ID NO: 6. In some embodiments, the linker comprises an amino acid sequence at least 30%, 35%, 40%, 45%, 50%, 55%, 60%, 65%, 70%, 75%, 80%, 85%, 90%, 95%, or 100% identical to SEQ ID NO: 8.

In some embodiments, the polypeptide further comprises an affinity/epitope tag. In some embodiments, the affinity/epitope tag comprises a polyhistidine tag, also referred herein as a His-Tag. In some embodiments, the His-Tag comprises a string of histidine residues. In some embodiments, the affinity/epitope tag is removed after the production and purification of the polypeptide.

In some embodiments, the polypeptide improves survival of cells associated with hematopoiesis in treated cells as compared to untreated cells. In some embodiments, the polypeptide stimulates proliferation of cells associated with hematopoiesis in treated cells as compared to untreated cells.

In some embodiments, the polypeptide activates MAPK pathway. In some embodiments, the polypeptide increases ERK phosphorylation in treated cells as compared to untreated cells. In some embodiments, the polypeptide increases Erk1/2 phosphorylation in treated cells as compared to untreated cells.

In some embodiments, the polypeptide activates PI3K/Akt pathway. In some embodiments, the polypeptide increases Akt phosphorylation in treated cells as compared to untreated cells. In some embodiments, the polypeptide increases Akt phosphorylation in treated cells as compared to untreated cells.

In some embodiments, the one or more alterations of the Fc domain as compared to the wild type IgG Fc region affects an immunological property of the Fc domain. In some embodiments, the immunological property comprises antigen-dependent cellular cytotoxicity (ADCC), complement dependent cytotoxicity (CDC), and antibody-dependent cell-mediated phagocytosis (ADCP), or a combination thereof. In some embodiments, the immunological property comprises ADCC. In some embodiments, the immunological property comprises CDC. In some embodiments, the immunological property comprises ADCP.

### Therapeutic Methods

Provided herein are methods of supporting cancer treatment in an individual comprising administering the polypeptides provided herein to the individual. Provided herein are methods of treating thrombocytopenia, leukopenia, neutropenia, anemia, and other blood-related side effects associated with a cancer treatment in an individual comprising administering the compositions provided herein to the individual. Such cancer treatments may affect the bone marrow and result in reduced production of one or more blood-related cells, including but not limited to red blood cells, white blood cells, platelets, and neutrophils. In some cases, the cancer treatments may result in reduced production of one or more cells of myeloid, lymphoid, or hematopoietic lineages. Provided herein are methods of increasing the production of one or more of red blood cells, white blood cells, platelets, or neutrophils in an individual undergoing a cancer treatment comprising administering the compositions provided herein to the individual. Provided herein are methods of increasing the production of one or more cells of myeloid, lymphoid, or hematopoietic lineages in an individual undergoing a cancer treatment comprising administering the compositions provided herein to the individual. Such cancer treatments may include but are not limited to chemotherapy, radiation therapy, immunotherapy, radiofrequency ablation, cryoablation, bone marrow transplantation, targeted drug therapy, and cell-based therapies.

Provided herein are methods of modulating an immune response in an individual comprising administering the polypeptides provided herein to the individual. In some embodiments, modulating an immune response in an individual includes but is not limited to activation and promoting infiltration of T cells, B cells, NK cells, dendritic cells and other innate immune cells in a tumor or infection. Provided herein are methods of modulating an immune response in an individual comprising administering the compositions provided herein to the individual. In some embodiments, the individual may be receiving treatments that include but are not limited to chemotherapy, radiation therapy, immunotherapy, radiofrequency ablation, cryoablation, bone marrow transplantation, and targeted drug therapy. In some embodiments, the treatment comprises Irreversible Electroporation (IRE), Microwave, Low-Intensity Focused Ultrasound (LOFU), High-Intensity Focused Ultrasound (HIFU), Radiofrequency energy, or cryotherapy or a combination thereof. In some embodiments, the polypeptide is administered in combination with the treatment. In some embodiments, the polypeptide is administered before the treatment. In some embodiments, the polypeptide is administered after the treatment. In some embodiments, the polypeptides are administered within 1 hour, 6 hours, 12 hours, 1 day, 2 days, 3 days, 4 days, 5 days, 6 days, 1 week, 2 weeks, 3 weeks, or 4 weeks of the treatment. In some embodiments, the polypeptides are administered 1 hour, 6 hours, 12 hours, 1 day, 2 days, 3 days, 4 days, 5 days, 6 days, 1 week, 2 weeks, 3 weeks, or 4 weeks before the treatment. In some embodiments, the polypeptides are administered 1 hour, 6 hours, 12 hours, 1 day, 2 days, 3 days, 4 days, 5 days, 6 days, 1 week, 2 weeks, 3 weeks, or 4 weeks after the treatment.

In some embodiments, the polypeptide stimulates immune response against tumors in synergy with the treatment. In some embodiments, the administration of the polypeptide includes at least 2, 3, 4, 5, 6, 7, 8, 9, or 10 doses of administration. In some embodiments, the doses are spaced a part by at least 6 hours, days, or even weeks.

Provided herein are methods of activating dendritic cells comprising administering the polypeptides provided herein to the individual. Provided herein are methods of increasing proliferation and activation of dendritic cell precursors and mature cells comprising administering the polypeptides provided herein to the individual.

In some embodiments, the administration of the polypeptide to a subject stimulates a platelet production in the subject. In some embodiments, the administration of the polypeptide to a subject stimulates a platelet production in the subject as compared to before the administration. In some embodiments, the administration of the polypeptide to a subject stimulates a platelet production in the subject as compared to the administration of a polypeptide comprising an FLT3L domain and a Fc domain without a thrombopoietin domain. In some embodiments, the administration of the polypeptide to a subject stimulates a platelet production in the subject similarly as the administration of a polypeptide comprising a thrombopoietin domain. In some embodiments, the administration of the polypeptide to a subject increases a number of platelets in the subject. In some embodiments, the administration of the polypeptide to a subject increases the number of platelets in the subject as compared to before the administration. In some embodiments, the administration of the polypeptide to a subject increases the number of platelets in the subject as compared to the administration of a polypeptide comprising an FLT3L domain and a Fc domain without a thrombopoietin domain. In some embodiments, the administration of the polypeptide to a subject increases the number of platelets in the subject similarly as the administration of a polypeptide comprising a thrombopoietin domain.

In some embodiments, an administration of the polypeptide to a subject increases a number of dendritic cells in a spleen in the subject. In some embodiments, an administration of the polypeptide to a subject increases a number of dendritic cells in blood of the subject. In some embodiments, the administration of the polypeptide increases dendritic cell maturation. In some embodiments, the administration of the polypeptide to a subject increases dendritic cell activation in the subject. In some embodiments, the administration of the polypeptide to a subject increases dendritic cell maturation in the subject. In some embodiments, the administration of the polypeptide to a subject increases dendritic cell expansion in the subject.

In some embodiments, the one or more alterations of the Fc domain as compared to the wild-type IgG Fc region affects one or more of the pharmacokinetic properties and/or pharmacodynamic properties of the fusion polypeptide. In some embodiments, the one or more alterations of the Fc domain as compared to the wild-type IgG Fc region increases one or more of the pharmacokinetic properties and/or pharmacodynamic properties of the fusion polypeptide. In some embodiments, the one or more alterations of the Fc domain as compared to the wild-type IgG Fc region decreases one or more of the pharmacokinetic properties and/or pharmacodynamic properties of the fusion polypeptide. In some embodiments, the one or more alterations of the Fc domain as compared to the wild-type IgG Fc region maintains one or more of the pharmacokinetic properties and/or pharmacodynamic properties of the fusion polypeptide. In some embodiments, the pharmacokinetic properties comprises bioavailability. In some embodiments, the pharmacodynamic properties comprises platelet production. In some embodiments, the pharmacodynamic properties comprises dendritic cell maturation, activation, production, and/or expansion.

In some embodiments, the bioavailability is measured by plasma concentration at various time points. In some embodiments, the various time points comprise one or more of 1, 2, 3, 4, 5, 6, 7, 8, 9, 10, 11, 12, 13, or 14 days. In some embodiments, the various time points comprise 1, 2, 3, 4, 5, 6, 7, 8, 9, 10, 11, 12, 13, or 14 days. In some embodiments, the various time points comprise one or more of 1, 2, 3, or 4 weeks. In some embodiments, the various time points comprise 4 and 7 days. In some embodiments the various time points comprise 14 days. In some embodiments, the various time points are immediately after administration. In some embodiments, the various time points comprise 1, 2, 3, 4, 5, 6, 7, 8, 9, 10, 11, 12, 13, 14, 15, 16, 17, 18, 19, 20, 21, 22, 23, or 24 hours after administration. In some embodiments, the one or more alterations of the Fc domain fusion polypeptide leads to increased bioavailability via intravenous injection compared to subcutaneous injection. In some embodiments, the one or more alterations of the Fc domain fusion polypeptide leads to increased bioavailability compared to thrombopoietin. In some embodiments, the one or more alterations of the Fc domain fusion polypeptide leads to a comparable bioavailability to FLT3L-Fc fusion polypeptide. In some embodiments, the change in bioavailability is measured after 1, 2, 3, 4, 5, 6, 7, 8, 9, 10. 11, 12, 13, 14, 15, 16, 17, 18, 19, 20, or 21 days after administration.

In some embodiments, the FLT3L plasma concentration is measured to determine one or more pharmacokinetic and/or pharmacodynamic properties. In some embodiments, the FLT3L plasma concentration is at least 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1500 pg/ml, 2000 pg/ml, 3000 pg/ml, 4000 pg/ml, 5000 pg/ml, or 6000 pg/ml. In some embodiments, the FLT3L plasma concentration is at most 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1500 pg/ml, 2000 pg/ml, 3000 pg/ml, 4000 pg/ml, 5000 pg/ml, or 6000 pg/ml. In some embodiments, the FLT3L plasma concentration is about 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1500 pg/ml, 2000 pg/ml, 3000 pg/ml, 4000 pg/ml, 5000 pg/ml, or 6000 pg/ml. In some embodiments, the FLT3L plasma concentration is measured on one or more of day 1, 2, 3, 4, 5, 6, 7, 8, 9, 10, 11, 12, 13, 14, 15, 16, 17, 18, 19, 20, or 21 after the administration. In some embodiments, the FLT3L plasma concentration at day 4 is at least 400 pg/ml. In some embodiments, the plasma concentration at day 4 is at least 2000 pg/ml. In some embodiments, the plasma concentration at day 4 is at least 6000 pg/ml. In some embodiments, the plasma concentration at day 4 is at most 400 pg/ml. In some embodiments, the plasma concentration at day 4 is at most 2000 pg/ml. In some embodiments, the plasma concentration at day 4 is at most 6000 pg/ml. In some embodiments, the plasma concentration at day 4 is about 400 pg/ml. In some embodiments, the plasma concentration at day 4 is about 2000 pg/ml. In some embodiments, the plasma concentration at day 4 is about 6000 pg/ml.

In some embodiments, when a thrombopoietin domain binds to a TPO receptor, the thrombopoietin domain may lead to increased platelet production. In some embodiments, the fusion polypeptides comprising a thrombopoietin domain described herein lead to increased platelet production or platelet numbers in a subject after administration. In some embodiments, the platelet production is measured by platelet counts. In some embodiments, platelet counts are collected by sampling the subject's blood at various time points. In some embodiments, the various time points comprise day 1, 2, 3, 4, 5, 6, 7, 8, 9, 10, 11, 12, 13, 14, 15, 16, 17, 18, 19, 20, or 21 after the administration, or a combination thereof. In some embodiments, the various time points are 4 and 6 days. In some embodiments, the various time points are 4 and 7 days. In some embodiments, the one or more alterations of the Fc domain fusion polypeptide leads to increased platelet counts over controls. In some embodiments, the one or more alterations of the Fc domain fusion polypeptide leads to increased platelet counts over FLT3L-Fc. In some embodiments, the one or more alterations of the Fc domain fusion polypeptide leads to a comparable increase in platelet counts to Romiplostim after 4 days.

In some embodiments, the platelet counts increase by at least 1, 1.5, 2, 2.5, 3, 3.5, 4, 4.5, 5 fold over a control. In some embodiments, the platelet counts are taken on one or more of day 1, 2, 3, 4, 5, 6, 7, 8, 9, 10, 11, 12, 13, 14, 15, 16, 17, 18, 19, 20, or 21 after the administration. In some embodiments, the platelet counts are increased at least 1.5 fold over controls after 4 days. In some embodiments, the platelet counts are increased at least 2 fold over controls after 4 days. In some embodiments, the platelet counts are increased by at least 150000; 200000; 300000; 400000; 500000; 600000; 700000; 800000; 900000; 1000000, or 2000000 platelets per microliter over a control. In some embodiments, the platelet counts are increased to at least 1500000 platelets per microliter after 4 days. In some embodiments, the platelet counts are increased to at least 2000000 platelets per microliter after 4 days. In some embodiments, the platelet counts increase by at least 10%, 20%, 30%, 40%, 50%, 60%, 60\hat{ }, 80%, 90%, or 100% over a control. In some embodiments, the platelet counts increase by at least 50% over controls after 4 days. In some embodiments, the platelet counts increase by at least 100% over controls after 4 days.

In some embodiments, the platelet counts increase by at most 1, 1.5, 2, 2.5, 3, 3.5, 4, 4.5, 5 fold over a control. In some embodiments, the platelet counts are increased at most 1.5 fold over controls after 4 days. In some embodiments, the platelet counts are increased at most 2 fold over controls after 4 days. In some embodiments, the platelet counts are increased by at most 150000; 200000; 300000; 400000; 500000; 600000; 700000; 800000; 900000; 1000000, or 2000000 platelets per microliter over a control. In some embodiments, the platelet counts are increased to at most 1500000 platelets per microliter after 4 days. In some embodiments, the platelet counts are increased to at most 2000000 platelets per microliter after 4 days. In some embodiments, the platelet counts increase by at most 10%, 20%, 30%, 40%, 50%, 60%, 60\hat{ }, 80%, 90%, or 100% over a control. In some embodiments, the platelet counts increase by at most 50% over controls after 4 days. In some embodiments, the platelet counts increase by at most 100% over controls after 4 days.

In some embodiments, the platelet counts increase by about 1, 1.5, 2, 2.5, 3, 3.5, 4, 4.5, 5 fold over a control. In some embodiments, the platelet counts are increased by about 1.5 fold over controls after 4 days. In some embodiments, the platelet counts are increased by about 2 fold over controls after 4 days. In some embodiments, the platelet counts are increased by about 150000; 200000; 300000; 400000; 500000; 600000; 700000; 800000; 900000; 1000000, or 2000000 platelets per microliter over a control. In some embodiments, the platelet counts are increased to about 1500000 platelets per microliter after 4 days. In some embodiments, the platelet counts are increased to about 2000000 platelets per microliter after 4 days. In some embodiments, the platelet counts increase by about 10%, 20%, 30%, 40%, 50%, 60%, 60\hat{ }, 80%, 90%, or 100% over a control. In some embodiments, the platelet counts increase by about 50% over controls after 4 days. In some embodiments, the platelet counts increase by about 100% over controls after 4 days.

In some embodiments, the dendritic cell production is measured by percentage of dendritic cells (e.g., CD11c high and MHCII high) of CD45+ cells. In some embodiments, the percentage of dendritic cells of CD45+ cells are collected by sampling the patient's blood. In some embodiments, splenocytes are isolated from the sampling of the patient's blood. In some embodiments, unclotted blood is isolated from the sampling of the patient's blood. In some embodiments, splenic dendritic cells are measured from the isolated splenocytes. In some embodiments, blood dendritic cells are measured from the isolated unclotted blood. In some embodiments, the percentage of splenic dendritic cells of CD45+ cells is increased over controls. In some embodiments, the percentage of splenic dendritic cells of CD45+ cells is increased over thrombopoietin. In some embodiments, the percentage of blood dendritic cells of CD45+ cells is increased over controls. In some embodiments, the percentage of blood dendritic cells of CD45+ cells is increased over thrombopoietin. In some embodiments, the percentage of blood dendritic cells of CD45+ cells is comparable to FLT3L-Fc.

CD45 is a lymphocyte common antigen that is a receptor-linked protein tyrosine phosphatase that is expressed on leucocytes. In some embodiments, the percentage of spleen dendritic cells of CD45+ cells are increased at least 1, 2, 3, 4, 5, 6, 7, 8, 9, or 10 fold over a control. In some embodiments, the percentage of spleen dendritic cells of CD45+ cells are increased at least 8 fold over a control. In some embodiments, the percentage of spleen dendritic cells of CD45+ cells are increased at least 4 fold over a control. In some embodiments, the percentage of spleen dendritic cells of CD45+ cells are increased at least 2 fold over a control. In some embodiments, the percentage of spleen dendritic cells of CD45+ cells are increased to at least 1, 2, 3, 4, 5, 6, 7, 8, 9, or 10 percent. In some embodiments, the percentage of spleen dendritic cells of CD45+ cells are increased to at least 4 percent. In some embodiments, the percentage of spleen dendritic cells of CD45+ cells are increased to at least 4 percent. In some embodiments, the percentage of spleen dendritic cells of CD45+ cells are increased at least 100% over controls. In some embodiments, the percentage of spleen dendritic cells of CD45+ cells are increased at least 300% over controls. In some embodiments, the percentage of spleen dendritic cells of CD45+ cells are increased at least 500% over controls. In some embodiments, the percentage of spleen dendritic cells of CD45+ cells are increased at least 700% over controls.

In some embodiments, the percentage of spleen dendritic cells of CD45+ cells are increased at most 1, 2, 3, 4, 5, 6, 7, 8, 9, or 10 fold over a control. In some embodiments, the percentage of spleen dendritic cells of CD45+ cells are increased at most 8 fold over a control. In some embodiments, the percentage of spleen dendritic cells of CD45+ cells are increased at most 4 fold over a control. In some embodiments, the percentage of spleen dendritic cells of CD45+ cells are increased at most 2 fold over a control. In some embodiments, the percentage of spleen dendritic cells of CD45+ cells are increased to at most 1, 2, 3, 4, 5, 6, 7, 8, 9, or 10 percent. In some embodiments, the percentage of spleen dendritic cells of CD45+ cells are increased to at most 8 percent. In some embodiments, the percentage of spleen dendritic cells of CD45+ cells are increased to at most 4 percent. In some embodiments, the percentage of spleen dendritic cells of CD45+ cells are increased at most 100% over controls. In some embodiments, the percentage of spleen dendritic cells of CD45+ cells are increased at most 300% over controls. In some embodiments, the percentage of spleen dendritic cells of CD45+ cells are increased at most 500% over controls. In some embodiments, the percentage of spleen dendritic cells of CD45+ cells are increased at most 700% over controls.

In some embodiments, the percentage of spleen dendritic cells of CD45+ cells are increased about 1, 2, 3, 4, 5, 6, 7, 8, 9, or 10 fold over a control. In some embodiments, the percentage of spleen dendritic cells of CD45+ cells are increased by about 8 fold over a control. In some embodiments, the percentage of spleen dendritic cells of CD45+ cells are increased by about 4 fold over a control. In some embodiments, the percentage of spleen dendritic cells of CD45+ cells are increased by about 2 fold over a control. In some embodiments, the percentage of spleen dendritic cells of CD45+ cells are increased to about 1, 2, 3, 4, 5, 6, 7, 8, 9, or 10 percent. In some embodiments, the percentage of spleen dendritic cells of CD45+ cells are increased to about 8 percent. In some embodiments, the percentage of spleen dendritic cells of CD45+ cells are increased to about 4 percent. In some embodiments, the percentage of spleen dendritic cells of CD45+ cells are increased to about 100% over controls. In some embodiments, the percentage of spleen dendritic cells of CD45+ cells are increased to about 300% over controls. In some embodiments, the percentage of spleen dendritic cells of CD45+ cells are increased to about 500% over controls. In some embodiments, the percentage of spleen dendritic cells of CD45+ cells are increased to about 700% over controls.

In some embodiments, the percentage of blood dendritic cells of CD45+ cells are increased at least 10 fold over a control. In some embodiments, the percentage of blood dendritic cells of CD45+ cells are increased at least 5 fold over a control. In some embodiments, the percentage of blood dendritic cells of CD45+ cells are increased to at least 10 percent. In some embodiments, the percentage of blood dendritic cells of CD45+ cells are increased to at least 5 percent. In some embodiments, the percentage of blood dendritic cells of CD45+ cells are increased at least 100% over controls. In some embodiments, the percentage of blood dendritic cells of CD45+ cells are increased at least 400% over controls. In some embodiments, the percentage of blood dendritic cells of CD45+ cells are increased at least 900% over controls.

In some embodiments, the percentage of blood dendritic cells of CD45+ cells are increased at most 10 fold over a control. In some embodiments, the percentage of blood dendritic cells of CD45+ cells are increased at most 5 fold over a control. In some embodiments, the percentage of blood dendritic cells of CD45+ cells are increased to at most 10 percent. In some embodiments, the percentage of blood dendritic cells of CD45+ cells are increased to at most 5 percent. In some embodiments, the percentage of blood dendritic cells of CD45+ cells are increased at most 100% over controls. In some embodiments, the percentage of blood dendritic cells of CD45+ cells are increased at most 400% over controls. In some embodiments, the percentage of blood dendritic cells of CD45+ cells are increased at most 900% over controls.

In some embodiments, the percentage of blood dendritic cells of CD45+ cells are increased by about 10 fold over controls. In some embodiments, the percentage of blood dendritic cells of CD45+ cells are increased by about 5 fold over a control. In some embodiments, the percentage of blood dendritic cells of CD45+ cells are increased to about 10 percent. In some embodiments, the percentage of blood dendritic cells of CD45+ cells are increased to about 5 percent. In some embodiments, the percentage of blood dendritic cells of CD45+ cells are increased to about 100% over controls. In some embodiments, the percentage of blood dendritic cells of CD45+ cells are increased to about 400% over controls. In some embodiments, the percentage of blood dendritic cells of CD45+ cells are increased to about 900% over controls.

Provided herein are methods of increasing the viability, proliferation, and/or activation of cells associated with hematopoiesis in an individual undergoing a cancer treatment comprising administering the compositions provided herein to the individual. In some embodiments, the method improves survival of cells associated with hematopoiesis in treated cells as compared to untreated cells. In some embodiments, the method stimulates proliferation of cells associated with hematopoiesis in treated cells as compared to untreated cells. Cancer treatments described herein may result in reduced viability and/or proliferation of cells associated with hematopoiesis. In some embodiments, cells associated with hematopoiesis comprise hematopoietic stem cells. In some embodiments, the hematopoietic stem cells are CD34+. In some embodiments, cells associated with hematopoiesis are CD34+. In some embodiments, the cells associated with hematopoiesis may differentiate into one or more of red blood cells, white blood cells, platelets, or neutrophils. In some embodiments, the cells associated with hematopoiesis may differentiate into one or more of multipotent progenitors, common myeloid progenitor (CMP), common lymphoid progenitor (CLP), or hematopoietic mature cells.

In some embodiments, the method activates MAPK pathway. In some embodiments, the method increases ERK phosphorylation in treated cells as compared to untreated cells. In some embodiments, the method increases Erk1/2 phosphorylation in treated cells as compared to untreated cells. In some embodiments, the method activates PI3K/Akt pathway. In some embodiments, the method increases Akt phosphorylation in treated cells as compared to untreated cells. In some embodiments, the method increases ERK/Akt phosphorylation in treated cells as compared to untreated cells.

Provided herein are polypeptides comprising a thrombopoietin domain and a Flt3 ligand domain for use in a method of supporting cancer treatment in an individual.

In certain embodiments, disclosed herein, are polypeptides useful for the treatment of a cancer or tumor. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, peritoneal, and liver tumors. In certain embodiments, tumors which can be treated with the polypeptides of the disclosure comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Ewing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPoma, and Wilm's tumor. In certain embodiments, the tumor/cancer to be treated with one or more polypeptides of the disclosure comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the polypeptides of this disclosure comprises glioblastoma. In certain embodiments, the cancer treated with one or more polypeptides of this disclosure comprises pancreatic cancer. In certain embodiments, the cancer treated with one or more polypeptides of this disclosure comprises ovarian cancer. In certain embodiments, the cancer treated with one or more polypeptides of this disclosure comprises lung cancer. In certain embodiments, the cancer treated with one or more polypeptides of this disclosure comprises prostate cancer. In certain embodiments, the cancer treated with one or more polypeptides of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed.

In certain embodiments, the polypeptides can be administered to a subject in need thereof by any route suitable for the administration of polypeptides-containing pharmaceutical compositions, such as, for example, subcutaneous, intraperitoneal, intravenous, intramuscular, intratumoral, intracerebral, intraarterial, intrathecal, intracapsular, intraocular, intracardiac, intradermal, intraperitoneal, transtracheal, subcuticular, or intraarticular, etc. In certain embodiments, the polypeptides are administered intravenously. In certain embodiments, the polypeptides are administered subcutaneously. In certain embodiments, the polypeptides are administered intratumoral. In certain embodiments, the polypeptides are administered on a suitable dosage schedule, for example, weekly, twice weekly, monthly, twice monthly, once every two weeks, once every three weeks, or once a month etc. In certain embodiments, the polypeptides are administered once every three weeks. The polypeptides can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 1 mg/kg and about 20 mg/kg. In certain embodiments, the therapeutically acceptable amount is between about 1 mg/kg and about 10 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. Therapeutically effective amounts include amounts sufficient to ameliorate one or more symptoms associated with the disease or affliction to be treated.

### Compositions

Provided herein are compositions comprising the polypeptides provided herein and a pharmaceutically acceptable excipient. In some embodiments, the composition is formulated to be administered intravenously. In some embodiments, the composition is formulated to be administered either intravenously or subcutaneously or intra-tumorally.

In certain embodiments the polypeptides of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the polypeptide. Such compounds include salts, pH buffers, detergents, anti-coagulants, and preservatives. In certain embodiments, the polypeptides of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the polypeptide of the current disclosure can be shipped/stored lyophilized and reconstituted before administration. In certain embodiments, lyophilized polypeptide formulations comprise a bulking agent such as, mannitol, sorbitol, sucrose, trehalose, dextran 40, or combinations thereof. The lyophilized formulation can be contained in a vial comprised of glass or other suitable non-reactive material. The polypeptides when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the polypeptides described herein in a suitable container and one or more additional components selected from: instructions for use; a diluent, an excipient, a carrier, and a device for administration.

In certain embodiments, described herein is a method of preparing a treatment for deleterious effects of exposure to radiation exposure, such as hematopoietic failure, thrombocytopenia, and/or radiation syndrome, comprising admixing one or more pharmaceutically acceptable excipients, carriers, or diluents and a polypeptide of the current disclosure. In certain embodiments, described herein is a method of preparing a cancer treatment for storage or shipping comprising lyophilizing one or more polypeptides of the current disclosure.

### Polypeptide Expression and Production

Provided herein are nucleic acids encoding the polypeptides provided herein, comprising a thrombopoietin domain and a Flt3 ligand domain. Provided herein are nucleic acids encoding the polypeptides provided herein, comprising a romiplostim domain and a Flt3 ligand domain.

Provided herein are expression vector comprising the nucleic acids encoding the polypeptides comprising a thrombopoietin domain and a Flt3 ligand domain. Provided herein are cells comprising the nucleic acid encoding the polypeptides comprising a thrombopoietin domain and a Flt3 ligand domain. Provided herein are cells comprising the nucleic acid encoding the polypeptides comprising a romiplostim domain and a Flt3 ligand domain.

Provided herein are methods of producing a polypeptide comprising a thrombopoietin domain and a Flt3 ligand domain comprising culturing cells under conditions sufficient to express the polypeptide.

In some embodiments, various polypeptide expression systems may be transfected with the expression vector provided herein. In some embodiments, the expression system comprises a bacterial cell expression system, a yeast cell expression system, an insect cell expression system, or a mammalian cell expression system or a combination thereof. In some embodiments, the mammalian cell expression system comprises HEK293 or Chinese Hamster Ovary (CHO) cells or a combination thereof. In some embodiments, the insect cell expression system comprises SF9 or SF21 or a combination thereof. In some embodiments, the yeast cell expression system comprises Saccharomyces cerevisiae. In some embodiments, the bacterial cell expression system comprises E. coli. In some embodiments, the cells of the expression systems are cultured in a bioreactor.

In some embodiments, the polypeptide further comprises an affinity/epitope tag. In some embodiments, the affinity/epitope tag comprises a polyhistidine tag, also referred herein as a His-Tag. In some embodiments, the His-Tag comprises a string of histidine residues. In some embodiments, the affinity/epitope tag is removed after the production and purification of the polypeptide. In some embodiments, the polypeptide is secreted into the medium and purified from the medium. In some embodiments, the cells of the expression systems are lysed to access the polypeptide, and the lysate is processed to isolate the polypeptide. In some embodiments, the processing of the lysate includes but is not limited to washing, solubilization, and affinity chromatography.

### Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

A fms-related tyrosine kinase 3 ligand is also referred to as Flt3 ligand, Flt3L, Flt3-Ligand, Flt3-L, FLT3 ligand, FLT3L, FLT3-L, or FL. FL is a hematopoietic cytokine that is encoded by the FLT3LG gene in humans and is capable of binding to fms-like tyrosine kinase receptor Flt3/Flk2. FL has four helical bundles and is structurally homologous to stem cell factor (SCF) and colony stimulating factor 1 (CSF-1). There are multiple isoforms of FL, including but not limited to a transmembrane isoform and a membrane-bound isoform. The transmembrane isoform is about 209 amino acids (a.a.). A mature human Flt3 ligand has a 158 amino acid (a.a.) extracellular domain (ECD) with a cytokine-like domain and a juxtamembrane tether region, a 21 a.a. transmembrane segment, and a 30 a.a. cytoplasmic tail. The membrane-bound isoform can be proteolytically cleaved to generate a biologically active soluble isoform. In some embodiments, a FL domain of the fusion polypeptide provided herein comprises an isolated, a synthetic, or a recombinant polypeptide encoding for FL. In some embodiments, a FL domain of the fusion polypeptide provided herein comprises a FL polypeptide or a functional fragment thereof. In some embodiments, a function of the FL domain may be assessed by an ELISA or a FL performance assay, such as bead-based multiplex assays. (See e.g., Graddis T J, et al. J Biol Chem. 1998 Jul. 10; 273(28):17626-33).

A thrombopoietin is also referred herein as megakaryocyte growth and development factor, MGDF, or TPO. In some embodiments, a thrombopoietin domain herein refers to thrombopoietin or a functional fragment thereof, or romiplostim or a functional fragment thereof. Thrombopoietin (TPO) is a glycoprotein that in humans is encoded by the THPO gene. TPO is produced by the liver and kidney and regulates the production of platelets by stimulating the production and differentiation of megakaryocytes. TPO may be a ligand for MLP/C_MPL, the product of myeloproliferative leukemia virus oncogene. The plasma TPO level may be inversely correlated to the mass of megakaryocytes and platelets, which degrade the TPO following its binding to specific membrane receptors. In some embodiments, a function of a TPO or a TPO domain may be assessed by ELISA or a TPO performance assay, such as bead-based multiplex assays.

A domain used as herein may refer to a functional analog, a mimetic, or a synthetic bio-similar compound.

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific polypeptide” including grammatical equivalents refers to a bispecific molecule that preserves at least one fragment of a polypeptide able to specifically bind a target. A “multi-specific polypeptide” including grammatical equivalents refers to a multi-specific molecule that preserves at least one fragment of a polypeptide able to specifically bind with a target.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides, or comprise separate domains of the same polypeptide. A number of strategies may be used to covalently link molecules together. Linkers described herein may be utilized to join a FL domain and a Fc domain; or may be used to tether a thrombopoietin domain and a Fc domain; or the N- or C-terminus of the polypeptide to create a bispecific or multispecific binding molecule. These include but are not limited to polypeptide linkages between N- and C-termini of proteins or protein domains, linkage via disulfide bonds, linkage via chemical cross-linking reagents, and linkage via enzymatic coupling. In some cases, the enzymatic coupling comprises using Sortase A to install coupling partners, such as, but not limited to, click handles (azides and alkynes). In some cases, the enzymatic coupling comprises using formylglycine-generating enzyme (FGE) coupled with Hydrazino-iso-Pictet-Spengler (HIPS) chemistry. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length may be used. Useful linkers include glycine-serine polymers, including for example (GS)n, (GSGGS)n, (GGGGS)n, and (GGGS)n, where n is an integer of at least one, glycine-alanine polymers, alanine-serine polymers, and other flexible linkers. In some embodiments, the linker comprises a rigid linker, including but not limited to such as (EAAAK)n, where n is an integer of at least one. In some embodiments, the linker comprises a chimeric linker, including but not limited to GGGGS and EAAAK motifs. Exemplary linkers can include AAEPKSS, AAEPKSSDKTHTCPPCP, GGGG, GGGGGG, HPRGSG, GGGGSGGGGSGGGGSGGGGS, or GGGGDKTHTCPPCP. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers. In some embodiments, a linker having an appropriate length and flexibility may synergize the activation of multiple receptors. In some embodiments, this activation of multiple receptors can be advantageous when the cell number is very low in situations such as post radiation exposure.

The terms “polypeptide” and “protein” are used interchangeably to refer to a polymer of amino acid residues, and are not limited to a minimum length. Polypeptides, including the antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of a number of known techniques. For example, it may be desirable to improve the binding affinity and/or other biological properties of the polypeptides. Amino acid sequence variants of a polypeptide may be prepared by introducing appropriate modifications into the nucleotide sequence encoding the polypeptide, or by peptide synthesis. Such modifications include, for example, deletions from, and/or insertions into and/or substitutions of residues within the amino acid sequences of the polypeptide. Any combination of deletion, insertion, and substitution can be made to arrive at the final construct, provided that the final construct possesses the desired characteristics, e.g., target-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Alterations (e.g., substitutions) may be made to improve polypeptide affinity. Such alterations may be made in encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, or oligonucleotide-directed mutagenesis) can be used to improve polypeptide affinity (See e.g., Hoogenboom et al. in Methods in Molecular Biology 178:1-37 (2001)). Alternatively, or additionally, a crystal structure of a target-receptor complex to identify contact points between the target and the receptor. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include a polypeptide with an N-terminal methionyl residue. Other insertional variants of the molecule include the fusion to the N- or C-terminus of the polypeptide to an enzyme (e.g., for ADEPT) or a polypeptide which increases the serum half-life of the polypeptide. Examples of intrasequence insertion variants of the polypeptide molecules include an insertion of 3 amino acids in a chain. Examples of terminal deletions include a polypeptide with a deletion of 7 or less amino acids at an end of a chain.

In some embodiments, the fusion polypeptides are altered to increase or decrease their glycosylation (e.g., by altering the amino acid sequence such that one or more glycosylation sites are created or removed). A carbohydrate attached to an Fc region of a polypeptide may be altered. Native polypeptides from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in the polypeptide can be made, for example, to create polypeptide variants with certain improved properties. The polypeptide glycosylation variants can have improved ADCC and/or CDC function. In some embodiments, variants are provided having a carbohydrate structure that lacks fucose attached (directly or indirectly) to an Fc region. For example, the amount of fucose in such polypeptide may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn297 (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63(1):78-85). However, Asn₂₉₇ may also be located about ±3 amino acids upstream or downstream of position 297, i.e., between positions 294 and 300, due to minor sequence variations in polypeptides. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated polypeptides, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other glycosylation variants are also included (See e.g., U.S. Pat. No. 6,602,684).

In some embodiments, the fusion polypeptide provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the fusion polypeptide target. In some embodiments, the fusion polypeptide provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the fusion polypeptide target. The target can be an Flt3 receptor target. K_{D} can be measured by any suitable assay. In certain embodiments, KD can be measured using surface plasmon resonance assays (e.g., using a BIACORE®-2000, a BIACORE®-3000 or Octet).

In some embodiments, one or more amino acid modifications may be introduced into the Fc region of a polypeptide provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions. The Fc region variant may comprise a mouse Fc region sequence comprising an amino acid modification (e.g., a substitution) at one or more amino acid positions.

In some embodiments, one or more amino acid modifications may be introduced into the Fc region of an polypeptide provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions. The Fc region variant may comprise a mouse Fc region sequence comprising an amino acid modification (e.g., a substitution) at one or more amino acid positions.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the polypeptides described herein comprise the Fc regions selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIA_{H131}, and with lower affinity to FcγRIIA_{R131}FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1≥IgG3>>IgG4≥IgG2.

In some embodiments, the polypeptides of this disclosure are fused to or comprise an Fc region and possess one or more variants that possess reduced effector functions, which make it a desirable candidate for applications in which certain effector functions (such as complement fixation and ADCC) are unnecessary or deleterious. Such polypeptides can have decreased complement-dependent cytotoxicity (CDC), antibody-dependent cell cytotoxicity (ADCC), or antibody dependent cellular phagocytosis (ADCP). In some embodiments, the polypeptides of this disclosure comprise variants that possess increased effector functions for applications in which increased immunogenicity would be beneficial. Such polypeptides can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Fc-containing fusion polypeptides can have increased half-lives and improved binding to the neonatal Fc receptor (FcRn) (See e.g., US 2005/0014934). Such polypeptides can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, a polypeptide provided herein may be further modified to contain additional nonproteinaceous moieties that are known and available. The moieties suitable for derivatization of the polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the polypeptide may vary, and if two or more polymers are attached, they can be the same or different molecules.

The fusion polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the fusion polypeptides described herein can be used to infect, transfect, transform, or otherwise render a suitable cell transgenic for the nucleic acid, thus enabling the production of fusion polypeptides for commercial or therapeutic uses. Standard cell lines and methods for the production of Fc-comprising polypeptides from a large scale cell culture are known in the art. See e.g., Li et al., “Cell culture processes for monoclonal antibody production.” Mabs. 2010 September-October; 2(5): 466-477. In certain embodiments, the cell is a eukaryotic cell. In certain embodiments, the eukaryotic cell is a mammalian cell. In certain embodiments, the mammalian cell is a cell line useful for producing fusion polypeptides is a Chines Hamster Ovary cell (CHO) cell, an NS0 murine myeloma cell, or a PER. C6® cell. In certain embodiments, the nucleic acid encoding the fusion polypeptide is integrated into a genomic locus of a cell useful for producing fusion polypeptides. In certain embodiments, described herein is a method of making an fusion polypeptide comprising culturing a cell comprising a nucleic acid encoding an fusion polypeptide under conditions in vitro sufficient to allow production and secretion of said fusion polypeptide.

In certain embodiments, described herein, is a master cell bank comprising: (a) a mammalian cell line comprising a nucleic acid encoding a fusion polypeptide described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding a fusion polypeptide provided herein; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making fusion polypeptides described herein. Such methods comprise incubating a cell or cell-line comprising a nucleic acid encoding the fusion polypeptide in a cell culture medium under conditions sufficient to allow for expression and secretion of the fusion polypeptide, and further harvesting the polypeptides from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, size exclusion chromatography, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., polypeptides, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### EXAMPLES

The following illustrative examples are representative of embodiments of compositions and methods described herein and are not meant to be limiting in any way.

### Example 1: Expression of Fusion Polypeptides

The objective of this study was to produce and verify the Flt3 ligand-thrombopoietin fusion polypeptides produced in expression systems. The transfections were performed following standard protocol. Sequence verified FLT3L-Romiplostim Fc fusion midi DNA was transfected into Expi293™ expression system or FreeStyle™ expression system. For Expi293™ expression system, Enhancers I and II were added to the transfected culture on day 1. The culture was harvested on day 6, and the supernatant was purified using His60 Ni Superflow™ resin. The elution from Ni⁺² resin was concentrated and dialyzed against buffer (1×PBS+0.1 M Arginine+2% glycerol).

The endotoxin level in the polished and purified polypeptide was measured using Kinetic-QCL™ LAL assay using buffer as a diluent. The endotoxin concentration was 0.5 EU/ml. The final concentration of FLT3L-Romiplostim Fc fusion polypeptide from the Expi293™ expression system was 0.8 mg/ml.

The dialyzed samples were run on 4-12% Bis-Tris precast gel. The relevant lanes are as follows:
A: Unstained protein ladder (NEB; P7717)
B: FL-Romi Expi293™
D: FL-Romi Freestyle™
F: FL-Romi Expi293™
H: FL-Romi Freestyle™

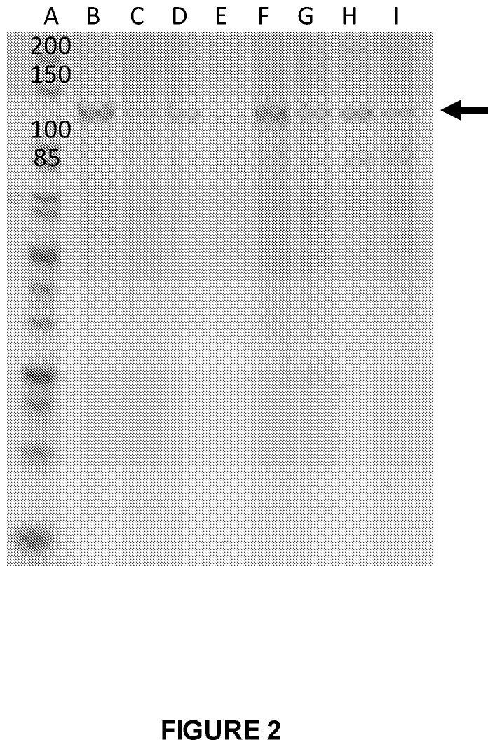

FIG. 2 illustrates a stained 4-12% Bis-Tris gel showing the expression of fusion polypeptides comprising a FL domain and a thrombopoietin domain in lanes B, D, F, and H. Lanes B, D, F, and H show a stained band (indicated with an arrow) between 100 and 150 kDa, indicating the expression of a fusion polypeptides comprising a FL domain and a thrombopoietin domain.

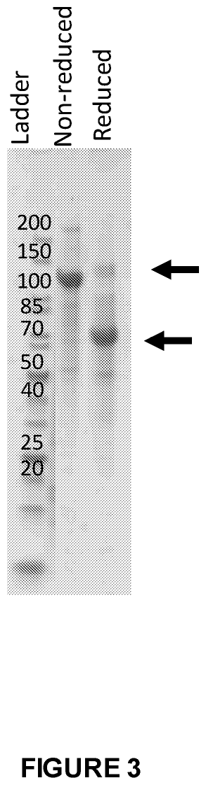

The dialyzed samples of the purified protein was reduced and ran on 4-12% Bis-Tris precast SDS-PAGE gel. FIG. 3 illustrates the non-reduced and reduced purified FLT3L-1-Romiplostim protein on 4-12% Bis-Tris precast SDS-PAGE gel. SDS-PAGE gel that shows a protein band at approximately 100 kDa under non-reduced condition and approximately 70 kDa under reduced condition (indicated with an arrow), which corresponds to the predicted molecular weight of the FL-Romiplostim fusion polypeptide under each condition. The first lane shows the unstained protein ladder.

### Example 2: Effect of the Fusion Polypeptide on Apoptosis

To study the effect of the fusion polypeptide provided herein, a human myeloid leukemia cell line was cultured with the fusion polypeptide and assessed for rescue from apoptosis by flow cytometry. OCI-AML5 cell line, a human myeloid leukemia cell line which expresses the Flt3 receptor, was used for this study. The OCI-AML5 cells line was established from patients with acute myeloid leukemia (AML) and is constitutively growth factor-dependent. In some cases, the fusion polypeptides provided herein comprising a Flt3 ligand domain may stimulate cell proliferation and promote cell survival by inhibiting or reducing apoptosis through phosphorylation of MAPK and PI3K/Akt pathways. ERK and/or AKT polypeptides.

OCI-AML5 cells were seeded in serum free media without growth factors (0.5 million/ml/well) and cultured for 16 hours. The cells were grown for either 48 or 72 hours with the fusion polypeptide. The positive control was GM-CSF treated cells, and the negative control was cells treated without cytokines. OCI-AML5 cells were treated with fusion polypeptides, and positive control cells were treated with GM-CSF for 48 or 72 hours before analysis. The negative control treatment was media alone without any cytokines. The level of apoptosis was measured using Annexin V flow cytometry.

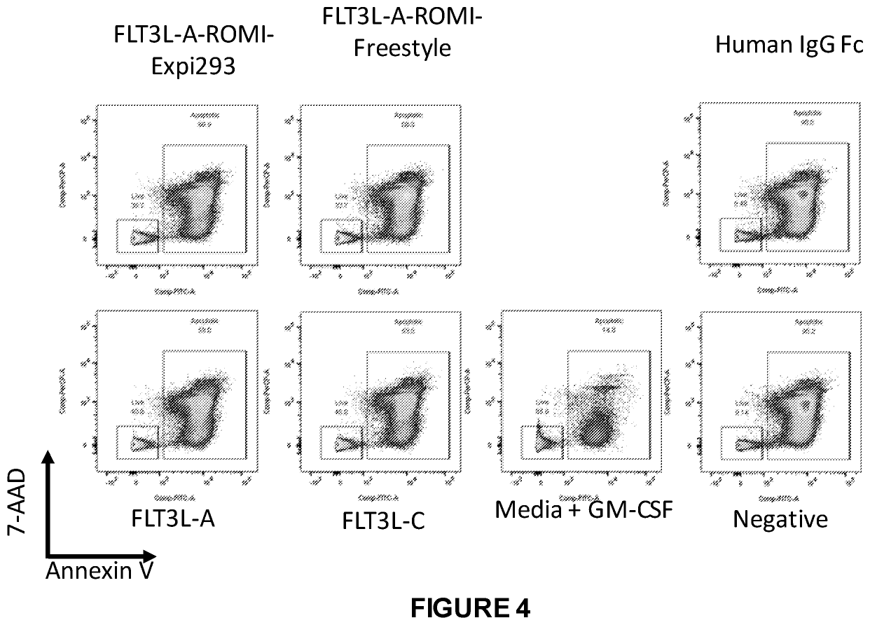

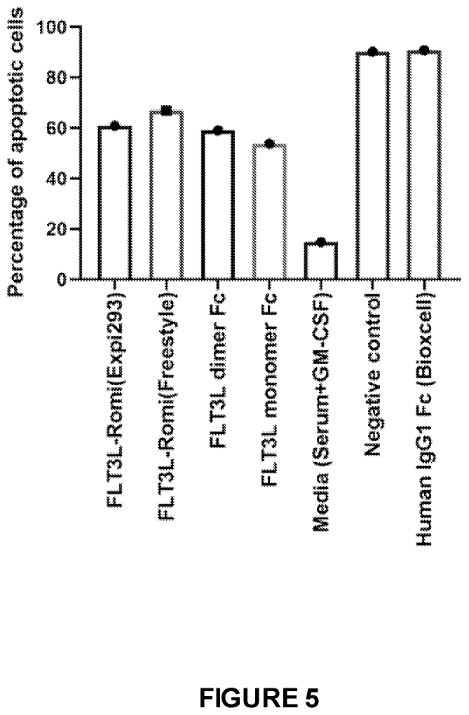

FIG. 4 shows the flow cytometry results showing the FL-romiplostim Fc fusion polypeptide treated OCI-AML5 cells. FIG. 5 shows the percentage of apoptotic cells from the flow cytometry results. For cells treated with FL-romiplostim Fc fusion polypeptide from Expi293 expression system, apoptosis was found in about 60.9% and had about 38.7% live cells. For cells treated with FL-romiplostim Fc fusion polypeptide from Freestyle expression system, apoptosis was found in about 66.9% and had about 32.7% live cells. For cells treated with FL dimer Fc fusion polypeptide, apoptosis was found in about 59.0% and had about 40.6% live cells. For cells treated with FL monomer Fc fusion polypeptide, apoptosis was found in about 53.8% and had about 45.8% live cells. For cells treated with media+CM-CSF (positive control), apoptosis was about 14.8% and had about 85.0% live cells. For negative control cells, apoptosis found in was about 90.2% and had about 9.14% live cells. For cells treated with human IgG1 Fc domain, about 90.8% of cells were indicative of apoptosis and about 8.48% of cells were live.

FL-romiplostim Fc fusion polypeptide treatment rescues OCI-AML5 cells from apoptosis.

### Example 3: Effect of the Fusion Polypeptide on MAPK Pathway

To study the effect of the fusion polypeptide provided herein, a human myeloid leukemia cell line was cultured with the fusion polypeptide and assessed for its effect on activation of MAPK pathway. OCI-AML5 cell line, a human myeloid leukemia cell line which expresses the Flt3 receptor, was used for this study. In some cases, the fusion polypeptides provided herein comprising a Flt3 ligand domain may stimulate cell proliferation and promote cell survival by inhibiting or reducing apoptosis and phosphorylating ERK and/or AKT polypeptides.

OCI-AML5 cells were seeded in serum free media (1 million/ml/well) and cultured without serum for 16 to 24 hours. Post serum starvation, the activation of MAPK kinase pathway by the added cytokine was measured using flow cytometry. The positive control was GM-CSF treated cells, and the negative control was cells treated without cytokines. OCI-AML5 cells were treated with the fusion polypeptides, and positive control cells were treated with GM-CSF for 3-5 minutes at 37° C. before analysis. The negative control treatment was media alone without serum and cytokines. The level of Erk1/2 phosphorylation was measured by flow cytometry using phospho-p44/42 MAPK (Erk1/2) (Thr202/Tyr204) antibody.

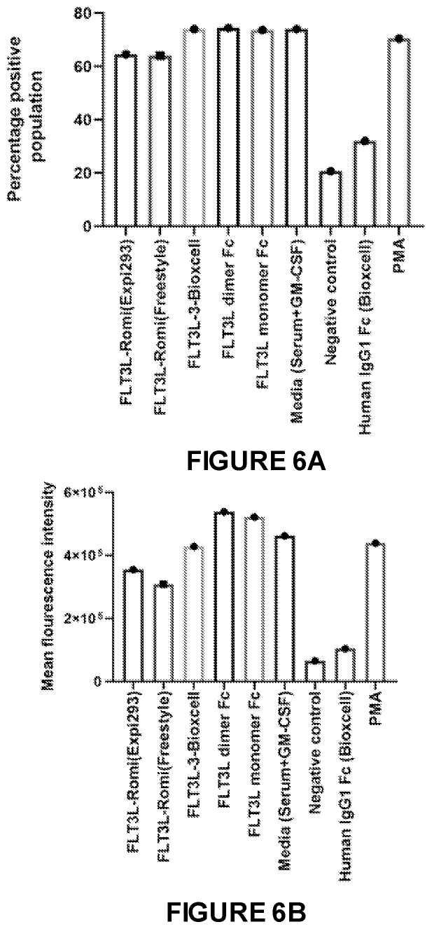

FIG. 6A shows the percentage of cells positive for Erk1/2 phosphorylation from the flow cytometry results. For cells treated with FL-romiplostim Fc fusion polypeptide from Expi293 expression system, about 62% of cells were positive for Erk1/2 phosphorylation. For cells treated with FL-romiplostim Fc fusion polypeptide from Freestyle expression system, about 62% of cells were positive for Erk1/2 phosphorylation. For cells treated with FL dimer Fc fusion polypeptide, about 75% of cells were positive for Erk1/2 phosphorylation. For cells treated with FL monomer Fc fusion polypeptide, about 75% of cells were positive for Erk1/2 phosphorylation. For cells treated with media+GM-CSF (positive control), about 75% of cells were positive for Erk1/2 phosphorylation. For negative control cells, about 20% of cells were positive for Erk1/2 phosphorylation. For cells treated with human IgG1 Fc domain, about 30% of cells were positive for Erk1/2 phosphorylation. FIG. 6B shows the mean fluorescence intensity for Erk1/2 phosphorylation from the flow cytometry results, which generally trend similarly with the percent positive cells of FIG. 6A.

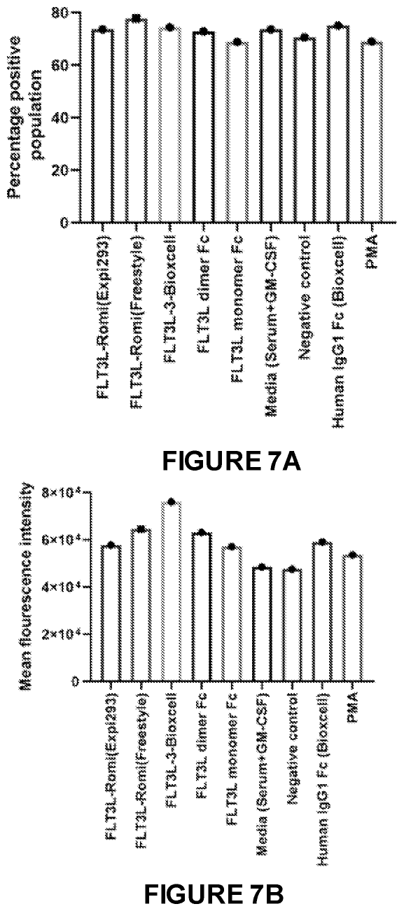

FIG. 7A shows the percentage of cells positive for Erk1/2 expression using p44/42 MAPK (Erk1/2) antibody from the flow cytometry results, providing a baseline for Erk1/2 expression of about 70-75% percent positive cells. FIG. 7B shows the mean fluorescence intensity using p44/42 MAPK (Erk1/2) antibody from the flow cytometry results, which generally trend similarly with the percent positive cells of FIG. 7A.

FL-romiplostim fusion polypeptide treatment of OCI-AML5 appears to activate MAPK pathway.

### Example 4: Effect of the Fusion Polypeptide on PI3K/Akt Pathway

To study the effect of the fusion polypeptide provided herein, a human acute megakaryoblastic leukemia cell line M-07e was cultured with the fusion polypeptide and assessed for its effect on activation of PI3K/Akt pathway. M-07e cell line expresses cMPL/TPO receptor, and the protein expression of Flt3 receptor is very low in M-07e cells. Therefore, M-07e cell line is optimal for testing the romiplostim function of the FL-romiplostim fusion polypeptide.

The M-07e cells were seeded in serum free media (1 million/ml/well) and cultured without serum for 16 hours. Post serum starvation, the activation of PI3K/Akt pathway by the added cytokine was measured using flow cytometry. The positive control was GM-CSF treated cells, and the negative control was cells treated without cytokines. M-07e cells were treated with the fusion polypeptide, and positive control cells were treated with GM-CSF for 3-5 minutes at 37° C. before analysis. The negative control treatment was media alone without serum and any cytokines. Akt phosphorylation was activated for 15 minutes with various growth factors. The level of PI3KAkt phosphorylation was measured by flow cytometry using Phospho-Akt (Ser473) antibody.

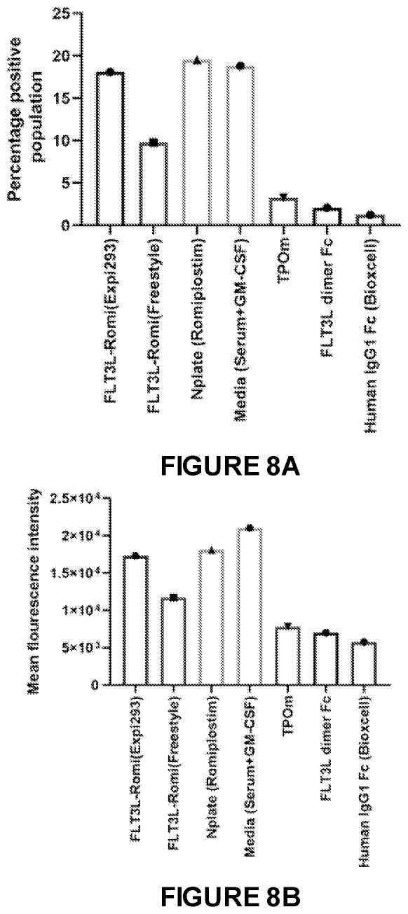

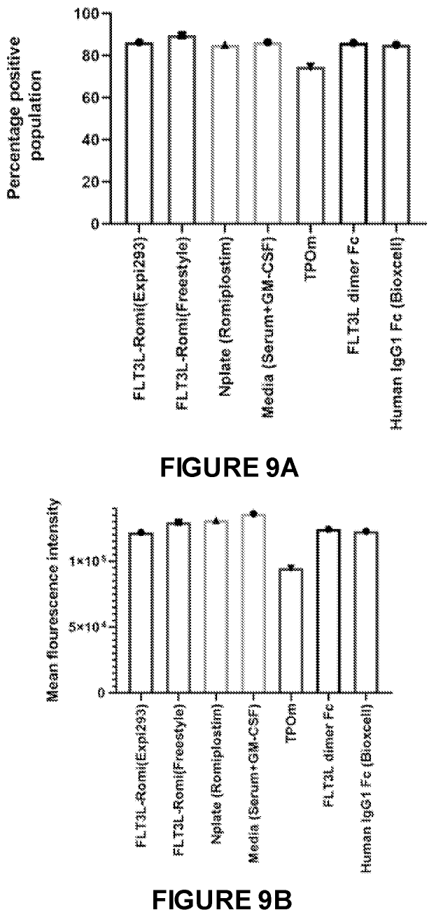

FIG. 8A shows the percentage of cells positive for PI3K/Akt phosphorylation from the flow cytometry results. For cells treated with FL-romiplostim Fc fusion polypeptide from Expi293 expression system, about 18% of cells were positive for Akt phosphorylation. For cells treated with FL-romiplostim Fc fusion polypeptide from Freestyle expression system, about 10% of cells were positive for Akt phosphorylation. For cells treated with romiplostim (Nplate), about 20% of cells were positive for Akt phosphorylation. For cells treated with media+CM-CSF (positive control), about 20% of cells were positive for Akt phosphorylation. For control cells treated with thrombopoietin mimetic (TPOm) peptide, less than 5% of cells were positive for Akt phosphorylation. For control cells treated with Flt3 ligand, less than 5% of cells were positive for Akt phosphorylation. For cells treated with human IgG1 Fc domain, less than 3% of cells were positive for Akt phosphorylation. FIG. 8B shows the mean fluorescence intensity for Akt phosphorylation from the flow cytometry results, which generally trend similarly with the percent positive cells of FIG. 8A. FIGS. 9A and B show the percentage of cells positive and mean fluorescence intensity for PI3K/Akt protein expression using Akt (pan) (C67E7) antibody from the flow cytometry results, providing a baseline for Akt expression of about 80-90% percent positive cells.

FL-romiplostim fusion polypeptide treatment of M-07e cells appears to activate PI3K/Akt pathway.

### Example 5: Effect of the Fusion Polypeptide on MAPK Pathway

To study the effect of the fusion polypeptide provided herein, a human acute megakaryoblastic leukemia cell line M-07e was cultured with the fusion polypeptide and assessed for its effect on activation of MAPK pathway. M-07e cell line expresses cMPL/TPO receptor, and the protein expression of Flt3 receptor is very low in M-07e cells. Therefore, M-07e cell line is optimal for testing the romiplostim function of the FL-romiplostim fusion polypeptide.

The M-07e cells were seeded in serum free media (1 million/ml/well) and cultured without serum and growth factor for 16-24 hours. Using flow cytometry, MAPK pathway activation via added cytokines was measured in serum starved M-07e cells. The positive control was GM-CSF treated cells, and the negative control was cells treated without serum and cytokines. M-07e cells were treated with the fusion polypeptide, and positive control cells were treated with GM-CSF for 3-5 minutes at 37° C. before analysis. The negative control treatment was media alone without serum and any cytokines. The level of ERK phosphorylation was measured by flow cytometry using Phospho-p44/42 MAPK (Erk1/2) (Thr202/Tyr204) antibody.

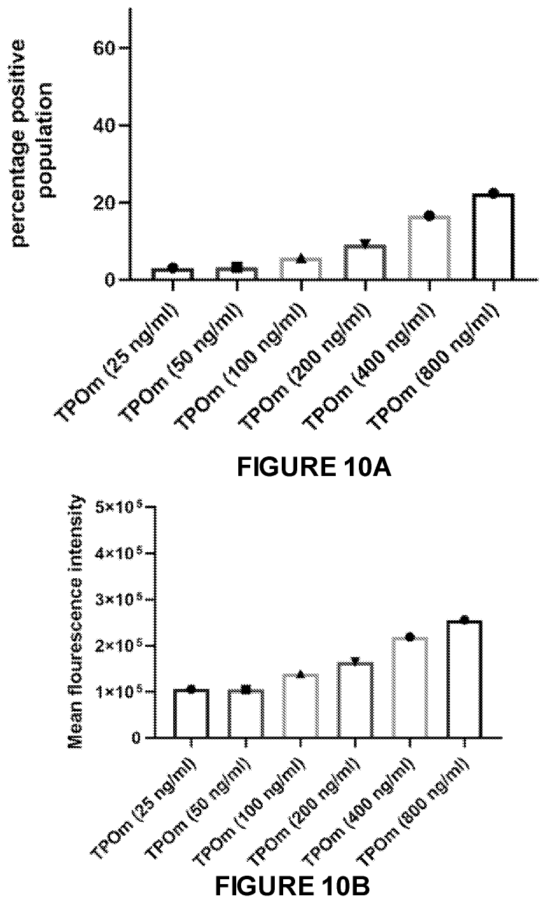

FIGS. 10A and B show the percentage of cells positive and mean fluorescence intensity for Erk1/2 phosphorylation after treatment with thrombopoietin mimetic (TPOm) peptide. The percentage of positive cells increases in a dose-dependent manner from 25 ng/ml TPOm to 800 ng/ml TPOm, from less than 5% percent positive to about 20% positive, respectively. The mean fluorescence intensities generally trended similarly with the percent positive cells of FIG. 10A, increasing in a TPOm dose-dependent manner. This shows that TPOm can activate the MAPK pathway.

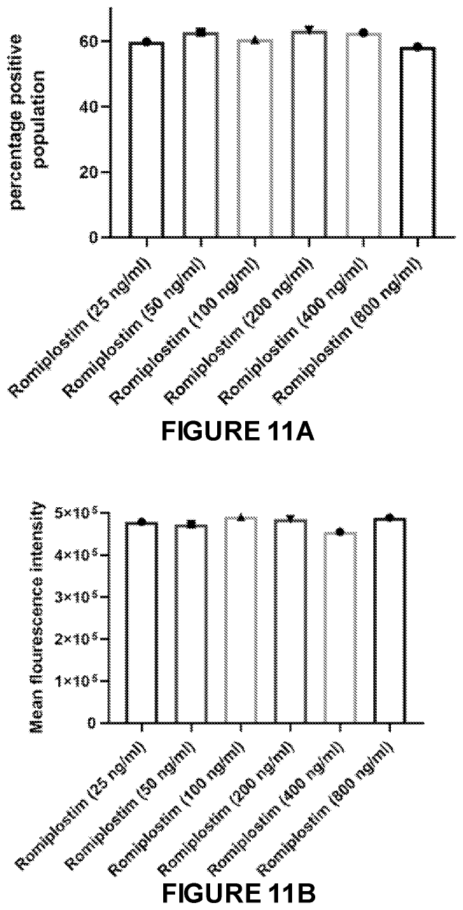

FIG. 11A shows the percentage of cells positive for Erk1/2 phosphorylation after treatment with romiplostim. The percentage of positive cells is consistently around 60% at different concentrations of romiplostim, ranging from 25 ng/ml romiplostim to 800 ng/ml romiplostim. This shows that romiplostim can activate the MAPK pathway. FIG. 11B shows the mean fluorescence intensity for Erk1/2 phosphorylation after treatment with romiplostim, which generally trended similarly with the percent positive cells of FIG. 11A.

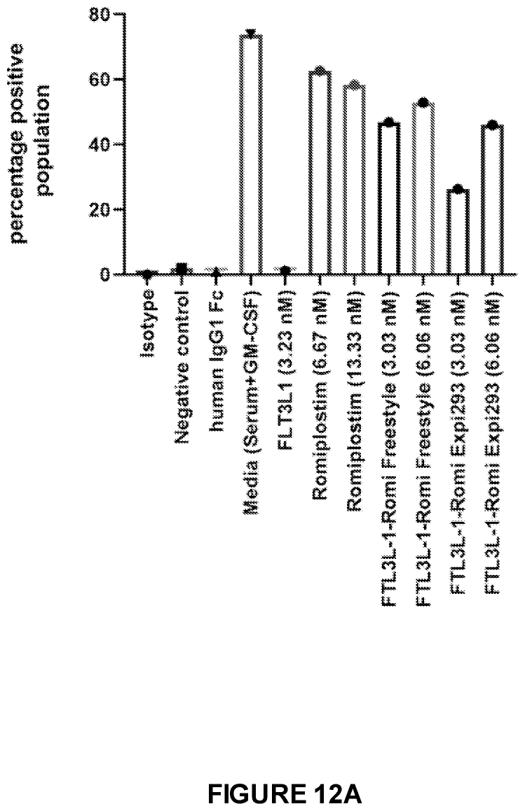

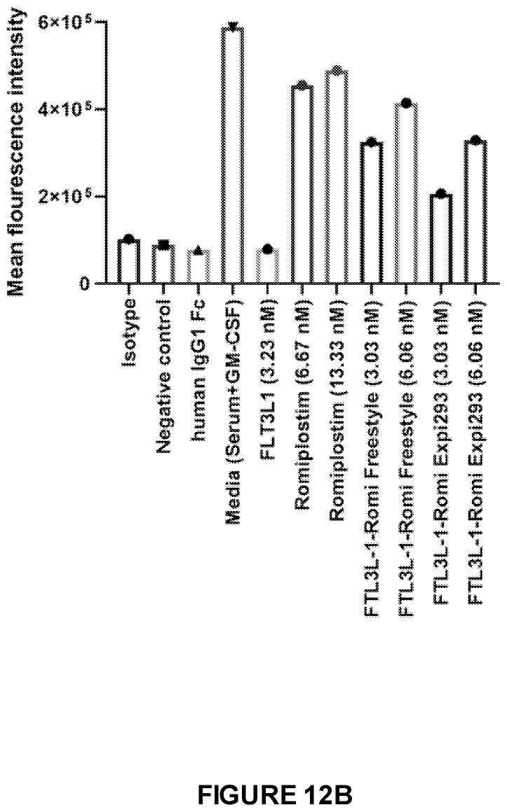

FIG. 12A shows the percentage of cells positive for ERK phosphorylation from the flow cytometry results. The cells treated with isotype, negative control, human IgG1 Fc, and FLT3L dimer Fc fusion had percentages of cells positive for Erk1/2 phosphorylation that were close to zero. For cells treated with media+GM-CSF (positive control), over 75% of cells were positive for Erk1/2 phosphorylation. For cells treated with romiplostim, about 60% of the cells were positive for Erk1/2 phosphorylation at 6.67 nM and 13.33 nM of romiplostim. For cells treated with FL-romiplostim Fc fusion polypeptide from Expi293 expression system, about 50% of cells were positive for Erk1/2 phosphorylation at 3.03 nM and about 55% were positive at 6.06 nM. For cells treated with FL-romiplostim Fc fusion polypeptide from Freestyle expression system, about 30% of cells were positive for Erk1/2 phosphorylation at 3.03 nM and about 50% were positive at 6.06 nM. FIG. 12B shows the mean fluorescence intensity for ERK phosphorylation from the flow cytometry results.

FL-romiplostim fusion polypeptide treatment of M-07e cells appears to activate MAPK pathway in a dose-dependent manner.

### Example 6: Effect of the Fusion Polypeptide on M-07e Cells

To study the effect of the fusion polypeptide provided herein, M-07e cell line was cultured with the fusion polypeptide and assessed for its effect on cell proliferation. The M-07e cells were seeded in serum free media (0.5 million/ml/well) and cultured without serum for 48 hours. After 48 hours of serum starvation, the cells were cultured with the FLT3L-romiplostim Fc fusion polypeptide or romiplostim for 72 hours. After the 72 hours of incubation, the proliferation of M-07e cells were measured using XTT assay.

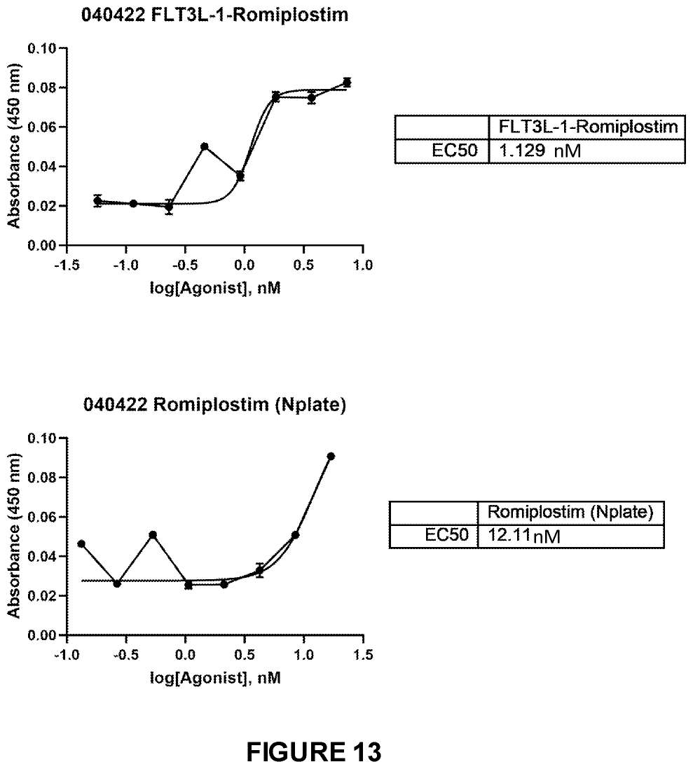

FIG. 13 shows the results of the XTT assay of the M-07e cell line cultured with the fusion polypeptide and assessed for its effect on cell proliferation. The absorbance is plotted against the log-scale of the concentration of the FLT3L-romiplostim Fc fusion polypeptide or romiplostim. The FLT3L-romiplostim fusion polypeptide treatment increased the proliferation of M-07e cells in a dose-dependent fashion with an EC50 value of 1.129 nM. The romiplostim treatment showed a similar trend and increased the proliferation of M-07e cells in a dose-dependent fashion, but with an EC50 value of 12.11 nM. The Flt3 ligand-romiplostim fusion polypeptide treatment of M-07e cells resulted an EC50 value that is more than 10-fold lower than the EC50 value for romiplostim treatment alone. The fusion polypeptide treatment demonstrated an improvement in cell proliferation than romiplostim alone.

### Example 6: Binding of the Fusion Polypeptide to Thrombopoietin Receptor cMPL

To study the binding capabilities of the fusion polypeptide provided herein to thrombopoietin receptors, the fusion polypeptides were contacted with cells expressing thrombopoietin receptor cMPL. Mouse or human thrombopoietin receptor cMPL was expressed on the surface of Freestyle™ HEK293 cell line. Mouse or human thrombopoietin receptor cMPL were expressed as a fusion polypeptide with either GPF or mCherry. Expression of cMPL receptors was confirmed by measuring fluorescent signal. As a general control, PD1 protein binding to PDL1-expressing cells was used. Human IgG1 Fc and secondary antibody were used as a negative control since they would not be expected to bind to cMPL. FLT3L-Dimer Fc was also used as a negative control to demonstrate binding of the fusion polypeptide is due to the Romiplostim. The cMPL expressing cells were treated with Nplate (Romiplostim) as the positive control. FLT3L-romiplostim fusion polypeptide with a His-Tag, FLT3L-romiplostim fusion polypeptide without a His-Tag were tested. Binding was analyzed using a fluorescent secondary antibody against IgG1 domain of the polypeptide. The percentages of cells bound to the tested polypeptide were quantified.

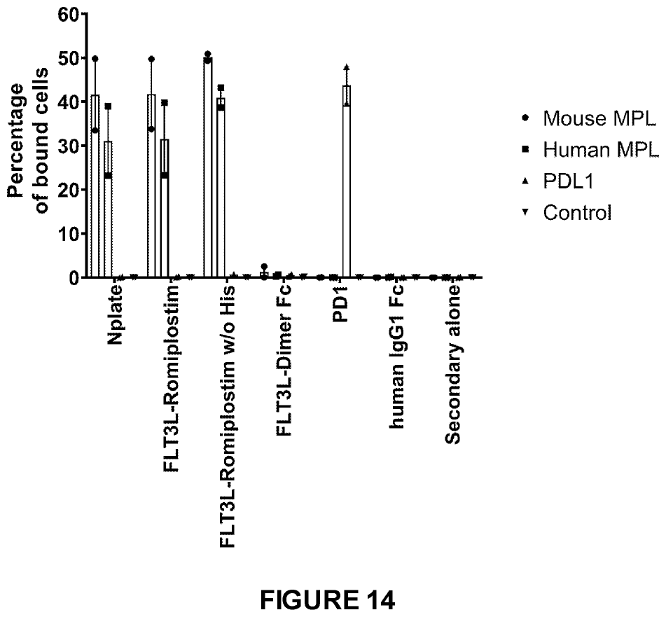

FIG. 14 shows the results of the binding of the tested polypeptides to the cMLP expressing Freestyle™ HEK293 cells. The percent of bound cells determined by secondary antibody fluorescent signal was plotted for each experimental condition. The FLT3L-Romiplostim with or without His-Tag showed comparable binding to mouse cMLP expressing cells as the positive control, Nplate (Romiplostim). The percentage of bound cells was above 40 percent in Nplate, FLT3L-Romiplostim with His tag and FLT3L-Romiplostim without His tag. The best performing condition was FLT3L-Romiplostim without His tag which had about 50 percent bound cells. The FLT3L-Romiplostim with or without His-Tag showed comparable binding to human cMLP expressing cells as the positive control, Nplate (Romiplostim). The percentage of bound cells was above 30 percent in Nplate, FLT3L-Romiplostim with His tag and FLT3L-Romiplostim without His tag. The best performing condition was FLT3L-Romiplostim without His tag which had about 40 percent bound cells. PDL1 binding with PD1 served as a general control and validated the assay format. There was no binding detected using negative controls, FLT3L-Dimer Fc, human IgG1 Fc, or secondary alone, as expected. The fusion polypeptide appears to bind both mouse and human cMLP.

### Example 7: Expression of Fusion Polypeptides Fc Mutants

The transfections were performed following standard protocol. Sequence verified FLT3L-Romiplostim Fc mutated fusion midi DNA was transfected into Expi-CHO expression system. For the expression system, Enhancers I and II were added to the transfected culture on day 1. The culture was harvested, and the supernatant was purified using Mab Select Sure column. The fusion polypeptides were further purified by size-exclusion chromatography.

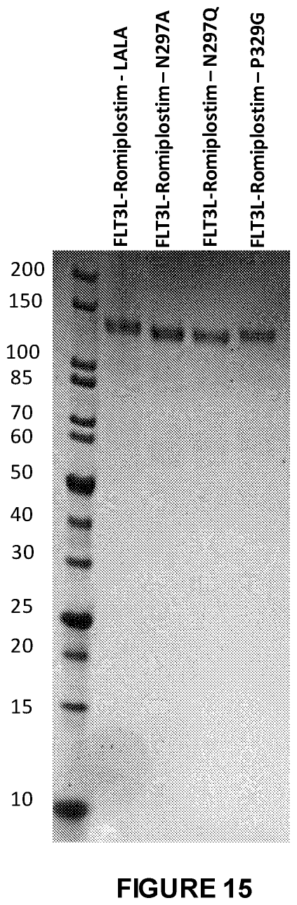

The dialyzed samples of the purified protein were reduced and ran on 4-12% Bis-Tris precast SDS-PAGE gel. FIG. 15 illustrates the purified FLT3L-Romiplostim Fc mutant polypeptides on 4-12% Bis-Tris precast SDS-PAGE gel. SDS-PAGE gel that shows protein bands at approximately 150 kDa for FLT3L-Romiplostim LALA, FLT3L-Romiplostim N297A, FLT3L-Romiplostim N297Q, and FLT3L-Romiplostim P329G, which corresponds to the predicted molecular weight of the FT3L-Romiplostim fusion polypeptide under each condition. The first lane shows the protein ladder.

### Example 8: Pharmacokinetics and Pharmacodynamics (PK/PD) of the Fusion Polypeptide

To study the pharmacokinetic and pharmacodynamic parameters of the fusion polypeptide Fc mutants provided herein, mice were treated with the fusion polypeptide and assessed for pharmacokinetics and pharmacodynamics. Mice were treated with a subcutaneous injection or intravenous administration of the fusion polypeptide at 0.1 mg/kg or 1 mg/kg. The vehicle for the injection was PBS, 0.1 M Arginine-cl, 2% glycerol. Mice were treated with FLT3L-Romiplostim with Fc mutations, romiplostim, FLT3 ligand Fc fusion from BioXcell, or vehicle (negative control). Table 1 shows the experimental design of the study. Samples of blood were taken to collect the plasma and platelets. The plasma was measured for Flt3 ligand using human Flt-3 Ligand/FLT3L DuoSet ELISA. The platelets were measured using a hemocytometer (Hemavet 950FS from Drew Scientific). Dendritic cells were isolated and analyzed using flow cytometry. Peripheral blood mononuclear cells and spleen cells were isolated and analyzed using flow cytometry for dendritic cell expansion. Bone-marrow was isolated and analyzed using flow cytometry for Megakaryocyte progenitors expansion.

TABLE 1
In Vivo Study Design

| | | | Mice per time | | |
|---|---|---|---|---|---|
| | Test | | point and total | Mice | Blood |
| Group | Article | Timing | mice per group | age | draw |
| 1 | FLT3L- | Day 4 and 7 | 1 | 15 weeks | Retro bleed (Day 4) |
| | Romiplostim | | | | and cardiac puncture |
| | without His-Tag | | | | (Day 7) |
| 2 | FLT3L- | Day 4 and 7 | 1 | 15 weeks | Retro bleed (Day 4) |
| | Romiplostim | | | | and cardiac puncture |
| | N297A | | | | (Day 7) |
| 3 | FLT3L- | Day 4 and 7 | 1 | 15 weeks | Retro bleed (Day 4) |
| | Romiplostim | | | | and cardiac puncture |
| | LALA | | | | (Day 7) |
| 4 | FLT3L- | Day 4 and 7 | 1 | 15 weeks | Retro bleed (Day 4) |
| | Romiplostim | | | | and cardiac puncture |
| | P329G | | | | (Day 7) |
| 5 | FLT3- | Day 4 and 7 | 1 | 15 weeks | Retro bleed (Day 4) |
| | Romiplostim | | | | and cardiac puncture |
| | N297Q | | | | (Day 7) |
| 6 | Romiplostim | Day 4 and 7 | 1 | 15 weeks | Retro bleed (Day 4) |
| | (Nplate) | | | | and cardiac puncture |
| | | | | | (Day 7) |
| 7 | FLT3 ligand Fc | Day 4 and 7 | 1 | 15 weeks | Retro bleed (Day 4) |
| | Fusion from | | | | and cardiac puncture |
| | BioXcell | | | | (Day 7) |
| 8 | PBS + 0.1M | Day 4 and 7 | 1 | 15 weeks | Retro bleed (Day 4) |
| | Ag-cl + 2% | | | | and cardiac puncture |
| | glycerol | | | | (Day 7) |
| | (Buffer) | | | | |

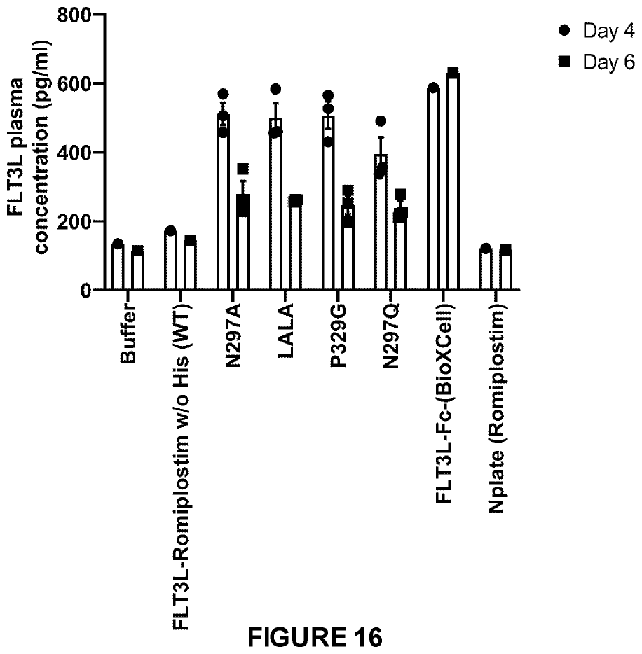

FIG. 16 illustrates the greater bioavailability of the four tested fusion polypeptide Fc mutants over FLT3L-Romiplostim without His-Tag. Mice were injected subcutaneously at 0.1 mg/kg. Buffer alone and Nplate were included as negative controls. The negative controls showed a baseline level of FLT3L plasma concentration of about 150 pg/m L at both day 4 and day 6. The FLT3L-Romiplostim without His tag had slightly elevated levels of FLT3L plasma concentration above baseline at about 175 pg/mL at Day 4 and 150 pg/mL at day 6. The FLT3L-Romiplostim Fc mutants had superior levels of FLT3L plasma concentration compared to baseline at both time points. At day 4, all four tested mutants, N297A, LALA (L234A and L235A), P329G, and N297Q had FLT3L plasma concentrations about or above 400 pg/mL. N297A, LALA, and P329G had FLT3L plasma concentrations at about 500 pg/m L. At day 6, all four tested mutants, N297A, LALA (L234A and L235A), P329G, and N297Q had FLT3L plasma concentrations above 200 pg/mL. A FLT3L-Fc fusion from BioXcell was included as a comparative positive control. The FLT3L-Fc fusion had a FLT3L plasma concentration at about 600 pg/mL on day 4 and about 650 pg/mL on day 6. These data demonstrated the improved bioavailability of the FLT3L-Romiplostim Fc mutants compared to FLT3L-Romiplostim without His tag wild-type Fc.

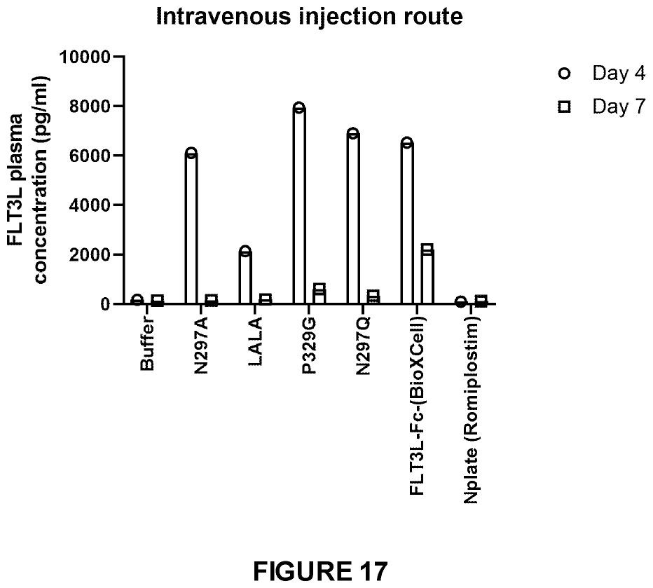

FIG. 17 illustrates the greater bioavailability of the four tested fusion polypeptide Fc mutants over baseline and a positive control, FLT3L-Fc fusion. Mice were injected intravenously at 1 mg/kg. Buffer alone and Nplate were included as negative controls. The negative controls showed a baseline level of FLT3L plasma concentration of about 0 pg/mL at both day 4 and day 6. The FLT3L-Romiplostim Fc mutants had superior levels of FLT3L plasma concentration compared to baseline at both time points. At day 4, all four tested mutants, N297A, LALA (L234A and L235A), P329G, and N297Q had FLT3L plasma concentrations about or above 2000 pg/mL. N297A, LALA, and P329G had FLT3L plasma concentrations at about or above 6000 pg/mL. P329G had FLT3L plasma concentrations at about 8000 pg/mL. At day 6, all four tested mutants, N297A, LALA (L234A and L235A), P329G, and N297Q had FLT3L plasma concentrations slightly above baseline. A FLT3L-Fc fusion from BioXcell was included as a comparative positive control. The FLT3L-Fc fusion had a FLT3L plasma concentration at about 6000 pg/mL on day 4 and about 2000 pg/mL on day 6. These data demonstrated the improved bioavailability of the FLT3L-Romiplostim Fc mutants compared to negative control and had similar plasma concentration as FLT3L-Fc fusion at day 4.

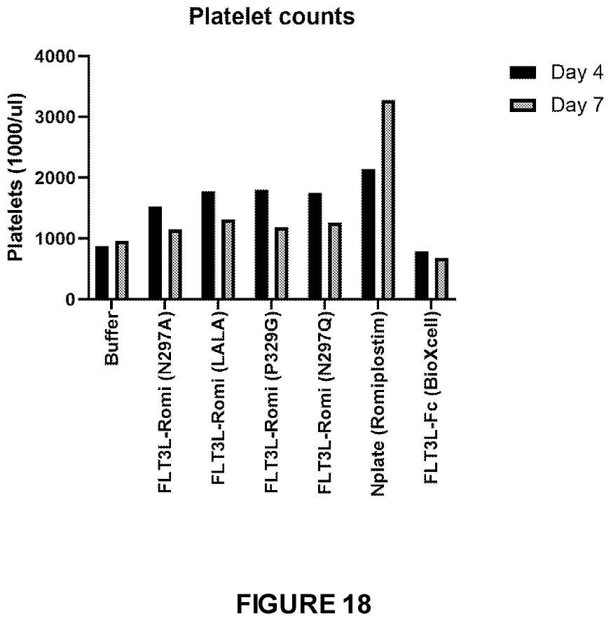

Platelet counts from blood drawn through retro-orbital route on day 4 and cardiac route on day 7 were analyzed using HEMA VET 950FS CBC machine. The in vivo functionality of the fusion polypeptide Fc mutants was illustrated in FIG. 18. All of the fusion polypeptides demonstrated similar increases in platelet counts on days 4 and 7. The buffer and FLT3L-Fc were included as negative controls. The negative controls showed a baseline level of platelets at about 1000000/μL The FLT3L-Romiplostim Fc mutants had superior levels of platelets compared to baseline at both time points. At day 4, all four tested mutants, N297A, LALA (L234A and L235A), P329G, and N297Q had FLT3L platelet concentrations about or above 1500000/μL. At day 6, all four tested mutants, N297A, LALA (L234A and L235A), P329G, and N297Q had FLT3L plasma concentrations slightly above baseline, at above 1000000 platelets/μL. Despite FLT3L-Romiplostim LALA having a lower bioavailability on day 7, the pharmacodynamics were greater than the other fusion polypeptides. Nplate (Romiplostim) was used a positive control. Nplate had platelet concentration of above 2000000 platelets/μL after day 4 and above 3000000 platelets/μL at day 7. The negative controls of buffer and FLT3L-Fc fusion from BioXcell both do not have a Romiplostim domain and demonstrated baseline platelet count levels. The positive control Nplate showed a rapid platelet increase. These data demonstrated the Romiplostim domain of the FLT3L-Romiplostim functions as intended as apparent from the increase of platelet concentrations at day 4 and day 7.

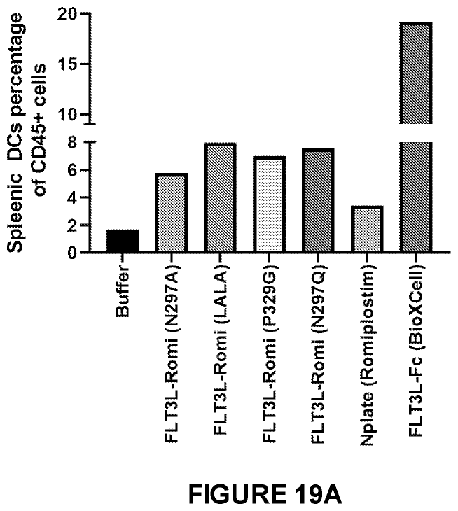

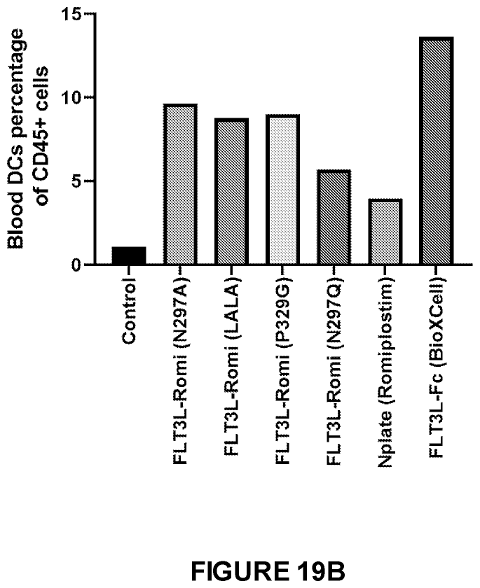

Splenocytes were isolated and analyzed using flow cytometry. Unclotted blood was labeled with antibodies, RBC were lysed using Cal-Lyse lysing solution and then analyzed using flow cytometry. The total dendritic cells (DCs) (CD11C high and MHC II high) were plotted as percentage of CD45+ cells. FIGS. 19A and 19B illustrates the percentage of DCs in both the spleen and blood of CD45+ cells. Buffer and Nplate (Romiplostim) were included as negative controls. For the splenic DC cells, the negative controls showed a baseline level of percentage of DCs pf CD45+ at about 2-3%. The FLT3L-Romiplostim Fc mutants had superior levels of percentage of spleen DC of CD45+ cells compared to baseline. All four tested mutants, N297A, LALA (L234A and L235A), P329G, and N297Q had percentage of spleen DC of CD45+ cells about or above 6 percent. LALA (L234A and L235A) had the highest of the fusion polypeptide Fc mutants, at about 8 percent. Despite FLT3L-Romiplostim LALA having a lower bioavailability on day 7, the pharmacodynamics were greater than the other fusion polypeptides. FLT3L-Fc fusion from BioXcell was used a positive control. FLT3L-Fc fusion had percentage of spleen DC of CD45+ cells about 20 percent. The negative controls of buffer and Nplate (Romiplostim) both do not have a FLT3L domain and demonstrated baseline spleen DC percentage of CD45+ cells. The positive control FLT3L-Fc fusion from BioXcell showed an increase in spleen DC percentage of CD45+ cells.

For the blood DC cells, the negative controls showed a baseline level of percentage of DCs of CD45+ at about 2-4%. The FLT3L-Romiplostim Fc mutants had superior levels of percentage of blood DC of CD45+ cells compared to baseline. All four tested mutants, N297A, LALA (L234A and L235A), P329G, and N297Q had percentage of spleen DC of CD45+ cells about or above 6 percent. N297A, LALA (L234A and L235A), and P329G had the highest of the fusion polypeptide Fc mutants at about 8 percent. Despite FLT3L-Romiplostim LALA having a lower bioavailability on day 7, the pharmacodynamics as great as the other fusion polypeptides. FLT3L-Fc fusion from BioXcell was used a positive control. FLT3L-Fc fusion had percentage of blood DC of CD45+ cells about 14 percent. The negative controls of buffer and Nplate (Romiplostim) both do not have a FLT3L domain and demonstrated baseline blood DC percentage of CD45+ cells. The positive control FLT3L-Fc fusion from BioXcell showed an increase in blood DC percentage of CD45+ cells. These data demonstrated the FLT3L domain of the FLT3L-Romiplostim functions as intended due to the increase from baseline, 2-4%, to about 6-8%.

The FLT3L-Romiplostim polypeptide Fc mutants demonstrated improved pharmacokinetic and pharmacodynamic properties than the FLT3L-Romiplostim without His-Tag polypeptide. The FLT3L-Fc fusion from BioXcell exceeded or matched the FLT3L-Romiplostim Fc mutant fusion polypeptide in FLT3L plasma concentration after day 4 or day 7 the production of DC cells in spleen and blood, but was far surpassed in mouse cMPL protein binding, human cMPL protein binding, platelet production. The Romiplostim exceeded or matched the FLT3L-Romiplostim Fc mutant fusion polypeptide in the production of platelets, but was far surpassed in FLT3L plasma concentration after day 4 or day 7 DC cell in spleen or blood production. Although individual parameters may not be increased over positive control, the overall parameters were improved in all stated categories.

### Example 8: Testing Treatment of FLT3L-Romiplostim Fusion Polypeptide in Acute Radiation Syndrome (ARS)

The objective of this study is to determine the rescue effect of FLT3L-Romiplostim P329G Fc mutant in acute radiation syndrome (ARS). It is hypothesized that the FLT3L-Romiplostim could protect or mitigate from ARS toxicity.

Experimental details: The polypeptide is injected subcutaneously into mice at 1 mg/kg one day before radiation (protection group) or one day post-radiation (mitigation group). The vehicle for injection is PBS, 0.1 M Arginine-cl, 2% glycerol. A negative control of vehicle alone is included. The mice are radiated using a partial body irradiation model. Unanesthetized C57BL/6J mice (9-11 weeks old from Jackson Laboratories) are restrained in 50-ml conical tubes with the left lower limb exteriorized outside the tube to shield the tibia, fibula, ankle, and foot under lead to provide 2.5% bone marrow shielding and then moved into the chamber of the CIX-3 orthovoltage irradiator (Xstrahl Inc., Suwanee, GA). This X-ray irradiator is operated at 300 k Vp, 10 mA with Thoraeus [4-mm Cu Half-Value Layer (HVL)] filtration and delivered at 1.12 Gy/min. The mice are exposed to 13 Gy PBI.

The mice are monitored for 30 days post-radiation for survival. The weight of the mice is taken to monitor radiation toxicity, which will provide information on the kinetics of the death, the reason for death, and drug pharmacodynamics. At the end time point, 50% of the negative control group are expected to succumb to death. It is also expected that the weight of the mice will be lower after 30 days in the negative control group. It is hypothesized that the protection group or mitigation group will have a lower percentage of mice succumbing to death than the negative control at 30 days. The weight of mice is also expected to have not decreased as greatly as the negative control group at 30 days.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the invention described herein may be employed in practicing the disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequence listings provided herein

| SEQ | | |
|---|---|---|
| ID NO: | Sequence | Origin |
| 1 | TQDCSFQHSPISSDFAVKIRELSDYLLQDYPVTVASNLQDEELCGG | Human FLT3 |
| | LWRLVLAQRWMERLKTVAGSKMQGLLERVNTEIHFVTKCAFQP | ligand domain- |
| | PPSCLRFVQTNISRLLQETSEQLVALKPWITRQNFSRCLELQCGGG | thrombopoietin |
| | GSGGGGSGGGGSGGGGSTQDCSFQHSPISSDFAVKIRELSDYLLQ | domain |
| | DYPVTVASNLQDEELCGGLWRLVLAQRWMERLKTVAGSKMQG | |
| | LLERVNTEIHFVTKCAFQPPPSCLRFVQTNISRLLQETSEQLVALKP | |
| | WITRQNFSRCLELQCQPHPRGSGEPKSCDKTHTCPPCPAPELLGGP | |
| | SVFLFPPKPKDTLMISRTPEVTCVVVDVSHEDPEVKFNWYVDGVE | |
| | VHNAKTKPREEQYNSTYRVVSVLTVLHQDWLNGKEYKCKVSNK | |
| | ALPAPIEKTISKAKGQPREPQVYTLPPSREEMTKNQVSLTCLVKGF | |
| | YPSDIAVEWESNGQPENNYKTTPPVLDSDGSFFLYSKLTVDKSRW | |
| | QQGNVFSCSVMHEALHNHYTQKSLSLSPGKGGGGGGIEGPTLRQ | |
| | WLAARAGGGGGGGGIEGPTLRQWLAARA | |
| 2 | MGVHECPAWLWLLLSLLSLPLGLPVL | EPO leader |
| | | sequence |
| 3 | TQDCSFQHSPISSDFAVKIRELSDYLLQDYPVTVASNLQDEELCGG | Human FLT3 |
| | LWRLVLAQRWMERLKTVAGSKMQGLLERVNTEIHFVTKCAFQP | ligand isoform 1 |
| | PPSCLRFVQTNISRLLQETSEQLVALKPWITRQNFSRCLELQC | (Membrane bound) |
| 4 | GGGGSGGGGSGGGGSGGGGS | Linker |
| 5 | TQDCSFQHSPISSDFAVKIRELSDYLLQDYPVTVASNLQDEELCGG | Human FLT3 |
| | LWRLVLAQRWMERLKTVAGSKMQGLLERVNTEIHFVTKCAFQP | ligand isoform 1 |
| | PPSCLRFVQTNISRLLQETSEQLVALKPWITRQNFSRCLELQCQP | (Membrane bound) |
| 6 | HPRGSG | Linker |
| 7 | EPKSCDKTHTCPPCPAPELLGGPSVFLFPPKPKDTLMISRTPEVTCV | Human IgG1 Fc |
| | VVDVSHEDPEVKFNWYVDGVEVHNAKTKPREEQYNSTYRVVSV | |
| | LTVLHQDWLNGKEYKCKVSNKALPAPIEKTISKAKGQPREPQVY | |
| | TLPPSREEMTKNQVSLTCLVKGFYPSDIAVEWESNGQPENNYKTT | |
| | PPVLDSDGSFFLYSKLTVDKSRWQQGNVFSCSVMHEALHNHYTQ | |
| | KSLSLSPGK | |
| 8 | GGGGGG | Linker |
| 9 | IEGPTLRQWLAARA | Thrombopoietin |
| | | domain |
| 10 | ENLYFQS | TEV cleavage |
| 11 | MGVHECPAWLWLLLSLLSLPLGLPVLLGTQDCSFQHSPISSDFAV | Human FLT3 |
| | KIRELSDYLLQDYPVTVASNLQDEELCGGLWRLVLAQRWMERLK | ligand domain- |
| | TVAGSKMQGLLERVNTEIHFVTKCAFQPPPSCLRFVQTNISRLLQE | thrombopoietin |
| | TSEQLVALKPWITRQNFSRCLELQCGGGGSGGGGSGGGGSGGGG | domain with EPO |
| | STQDCSFQHSPISSDFAVKIRELSDYLLQDYPVTVASNLQDEELCG | leader sequence |
| | GLWRLVLAQRWMERLKTVAGSKMQGLLERVNTEIHFVTKCAFQ | and His tag |
| | PPPSCLRFVQTNISRLLQETSEQLVALKPWITRQNFSRCLELQCQP | |
| | HPRGSGEPKSCDKTHTCPPCPAPELLGGPSVFLFPPKPKDTLMISRT | |
| | PEVTCVVVDVSHEDPEVKFNWYVDGVEVHNAKTKPREEQYNST | |
| | YRVVSVLTVLHQDWLNGKEYKCKVSNKALPAPIEKTISKAKGQP | |
| | REPQVYTLPPSREEMTKNQVSLTCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGGGGGIEGPTLRQWLAARAGGGGGGGGI | |
| | EGPTLRQWLAARAGGSENLYFQSGGSHHHHHHHHHH | |
| 12 | IEGPTLRQWLAARAGGSENLYFQSGGS | Thrombopoetin |
| | | domain with linker |
| | | and TEV cleavage |

What is claimed is:

1. A polypeptide comprising a thrombopoietin domain and a Flt3 ligand domain.

2. The polypeptide of claim 1, wherein the thrombopoietin domain comprises an amino acid sequence at least 80%, 85%, 90%, 95%, or 100% identical to SEQ ID NO: 9.

3. The polypeptide of claim 1, wherein the Flt3 ligand domain comprises an amino acid sequence at least 80%, 85%, 90%, 95%, or 100% identical to SEQ ID NO: 3 or 5.

4. A polypeptide comprising an amino acid sequence at least 80%, 85%, 90%, 95%, or 100% identical to SEQ ID NO: 1.

5. The polypeptide of any one of claims 1 to 3, wherein the polypeptide further comprises an immunoglobulin Fc polypeptide or a fragment thereof.

6. The polypeptide of claim 5, wherein the immunoglobulin is immunoglobulin G1 (IgG1).

7. The polypeptide of any one of claims 1 to 6, wherein the polypeptide further comprises an EPO leader sequence and/or a TEV cleavage domain.

8. The polypeptide of any one of claims 1 to 7, wherein the polypeptide further comprises a linker.

9. The polypeptide of any one of claims 1 to 8, wherein the linker couples the amino acid at least 80%, 85%, 90%, 95%, or 100% identical to SEQ ID NO: 9 to the immunoglobulin Fc polypeptide or a fragment thereof.

10. The polypeptide of any one of claims 1 to 9, wherein the linker couples the thrombopoietin domain to the immunoglobulin Fc polypeptide or a fragment thereof.

11. The polypeptide of any one of claims 1 to 10, wherein the linker couples the thrombopoietin domain to a second thrombopoietin domain.

12. The polypeptide of any one of claims 1 to 11, wherein the linker couples the TEV domain to the thrombopoietin domain or the second thrombopoietin domain.

13. The polypeptide of any one of claims 1 to 12, wherein the linker couples the amino acid at least 80%, 85%, 90%, 95%, or 100% identical to SEQ ID NO: 3 or 5 to the immunoglobulin Fc polypeptide or a fragment thereof.

14. The polypeptide of any one of claims 1 to 13, wherein the linker couples the Flt3 ligand domain to the immunoglobulin Fc polypeptide or a fragment thereof.

15. The polypeptide of any one of claims 1 to 14, wherein the Flt3 ligand domain is a Flt3 ligand isoform 1.

16. The polypeptide of any one of claims 1 to 15, wherein the Flt3 ligand domain is a human Flt3 ligand isoform 1.

17. The polypeptide of any one of claims 1 to 16, wherein the amino acid sequence is identical to SEQ ID NO: 1.

18. The polypeptide of any one of claims 4 to 17, wherein an immunoglobulin Fc polypeptide or a fragment thereof comprises one or more alterations compared to a wild type IgG Fc region as specified in SEQ ID NO: 7, wherein the one or more alterations affects an immunological property of the immunoglobulin Fc polypeptide or a fragment thereof.

19. The polypeptide of claim 18, wherein the one or more alterations comprises L234A, L235A, N297A, N297Q, P329Q, or a combination thereof according to EU numbering.

20. The polypeptide of claim 19, wherein the one or more alterations comprises L234A and L235A according to EU numbering.

21. The polypeptide of claim 19, wherein the one or more alterations comprises N297A according to EU numbering.

22. The polypeptide of claim 19, wherein the one or more alterations comprises N297Q according to EU numbering.

23. The polypeptide of claim 19, wherein the one or more alterations comprises P329Q according to EU numbering.

24. The polypeptide of any one of claims 18 to 23, wherein the immunological property comprises antigen-dependent cellular cytotoxicity (ADCC), complement dependent cytotoxicity (CDC), antibody-dependent cell-mediated phagocytosis (ADCP), or a combination thereof.

25. The polypeptide of any one of claims 1 to 24, wherein an administration of the polypeptide to a subject increases a number of platelets in the subject.

26. The polypeptide of any one of claims 1 to 24, wherein an administration of the polypeptide to a subject increases a number of dendritic cells in a spleen in the subject.

27. The polypeptide of any one of claims 1 to 24, wherein an administration of the polypeptide to a subject increases a number of dendritic cells in blood of the subject.

28. The polypeptide of any one of claims 1 to 27, wherein the polypeptide improves survival of cells associated with hematopoiesis in treated cells as compared to untreated cells.

29. The polypeptide of any one of claims 1 to 28, wherein the polypeptide stimulates proliferation of cells associated with hematopoiesis in treated cells as compared to untreated cells.

30. The polypeptide of any one of claims 1 to 29, wherein the polypeptide activates MAPK pathway.

31. The polypeptide of any one of claims 1 to 30, wherein the polypeptide increases ERK phosphorylation in treated cells as compared to untreated cells.

32. The polypeptide of any one of claims 1 to 31, wherein the polypeptide increases Erk1/2 phosphorylation in treated cells as compared to untreated cells.

33. The polypeptide of any one of claims 1 to 32, wherein the polypeptide activates PI3K/Akt pathway.

34. The polypeptide of any one of claims 1 to 33, wherein the polypeptide increases Akt phosphorylation in treated cells as compared to untreated cells.

35. The polypeptide of any one of claims 1 to 34, wherein the polypeptide increases ERK/Akt phosphorylation in treated cells as compared to untreated cells.

36. The polypeptide of any one of claims 1 to 35, wherein the polypeptide is configured to bind to a fms like tyrosine kinase 3 (FLT3).

37. A composition comprising the polypeptide of any one of claims 1 to 36 and a pharmaceutically acceptable excipient.

38. The composition of claim 37, wherein the composition is formulated to be administered either intravenously or subcutaneously or intra-tumorally.

39. A method of supporting cancer treatment in an individual, the method comprising administering the polypeptide of any one of claims 1 to 36 to the individual.

40. A method of supporting cancer treatment in an individual, the method comprising administering the composition of claim 37 or 38 to the individual.

41. A method of modulating an immune response in an individual, the method comprising administering the polypeptide of any one of claims 1 to 36 to the individual.

42. A method of stimulating the proliferation and activation of dendritic cells, the method comprising administering the polypeptide of any one of preceding claims to an individual.

43. The method of any one of claims 39 to 42, wherein the method improves survival of cells associated with hematopoiesis in treated cells as compared to untreated cells.

44. The method of any one of claims 39 to 43, wherein the method stimulates proliferation of cells associated with hematopoiesis in treated cells as compared to untreated cells.

45. The method of any one of claims 39 to 44, wherein the method activates MAPK pathway.

46. The method of any one of claims 39 to 45, wherein the method increases ERK phosphorylation in treated cells as compared to untreated cells.

47. The method of any one of claims 39 to 46, wherein the method increases Erk1/2 phosphorylation in treated cells as compared to untreated cells.

48. The method of any one of claims 39 to 47, wherein the method activates PI3K/Akt pathway.

49. The method of any one of claims 39 to 48, wherein method increases Akt phosphorylation in treated cells as compared to untreated cells.

50. The method of any one of claims 39 to 49, wherein the method increases ERK/Akt phosphorylation in treated cells as compared to untreated cells.

51. A nucleic acid encoding the polypeptide of any one of claims 1 to 36.

52. An expression vector comprising the nucleic acid of claim 51.

53. A cell comprising the nucleic acid encoding the polypeptide of any one of claims 1 to 36.

54. A method of producing a polypeptide of any one of claims 1 to 36 comprising culturing cells under conditions sufficient to express the polypeptide.

55. A polypeptide of any one of claims 1 to 36 for use in a method of supporting cancer treatment in an individual.
